SEQUENCE LISTING (56 sequences)

SEQ ID NO: 1 - protein, 15 aa, human
GGGGSGGGGSGGGGS

SEQ ID NO: 2 - protein, 15 aa, human
ESGRSGGGGSGGGGS

SEQ ID NO: 3 - protein, 14 aa, human
EGKSSGSGSESKST

SEQ ID NO: 4 - protein, 15 aa, human
EGKSSGSGSESKSTQ

SEQ ID NO: 5 - protein, 14 aa, human
EGKSSGSGSESKVD

SEQ ID NO: 6 - protein, 14 aa, human
GSTSGSGKSSEGKG

SEQ ID NO: 7 - protein, 18 aa, human
KESGSVSSEQLAQFRSLD

SEQ ID NO: 8 - protein, 16 aa, human
ESGSVSSEELAFRSLD

SEQ ID NO: 9 - DNA, 35 bp, human
tttgcggccgctcaggtgcarctgctcgagtcygg

SEQ ID NO: 10 - DNA, 66 bp, human
agatccgccgccaccgctcccaccacctccggagccaccgccacctgaggtgaccgtgaccrkggt

SEQ ID NO: 11 - DNA, 69 bp, human
ggtggcggtggctccggaggtggtgggagcggtggcggcggatctgagctcswgmtgacccagtctcca

SEQ ID NO: 12 - DNA, 47 bp, human
gggtctagactcgaggatccttattaacgcgttggtgcagccacagt

SEQ ID NO: 13 - protein, 6 aa, human
SEKDEL

SEQ ID NO: 14 - DNA, 59 bp, human
gggtctagactcgaggatccttattacagctcgtccttttcgcttggtgcagccacagt

SEQ ID NO: 15 - DNA, 24 bp, human
tttaccatggaacatctgtggttc

SEQ ID NO: 16 - DNA, 30 bp, human
ttagcgcgctgaggtgaccgtgaccrkggt

SEQ ID NO: 17 - protein, 4 aa, human
KDEL

SEQ ID NO: 18 - protein, 4 aa, human
DDEL

SEQ ID NO: 19 - protein, 4 aa, human
DEEL

SEQ ID NO: 20 - protein, 4 aa, human
QEDL

SEQ ID NO: 21 - protein, 4 aa, human
RDEL

SEQ ID NO: 22 - protein, 7 aa, human
PKKKRKV

SEQ ID NO: 23 - protein, 7 aa, human
PQKKIKS

SEQ ID NO: 24 - protein, 5 aa, human
QPKKP

SEQ ID NO: 25 - protein, 12 aa, human
RKKRRQRRRAHQ

SEQ ID NO: 26 - protein, 16 aa, human
RQARRNRRRRWRERQR

SEQ ID NO: 27 - protein, 19 aa, human
MPLTRRRPAASQALAPPTP

SEQ ID NO: 28 - protein, 15 aa, human
MDDQRDLISNNEQLP

SEQ ID NO: 29 - protein, 32 aa, human
  UNSURE (7)(8)(32): UNSURE
MLFNLRXXLNNAAFRHGHNFMVRNFRCGQPLX

SEQ ID NO: 30 - protein, 8 aa, human
GCVCSSNP

SEQ ID NO: 31 - protein, 8 aa, human
GQTVTTPL

SEQ ID NO: 32 - protein, 8 aa, human
GQELSQHE

SEQ ID NO: 33 - protein, 8 aa, human
GNSPSYNP

SEQ ID NO: 34 - protein, 8 aa, human
GVSGSKGQ

SEQ ID NO: 35 - protein, 8 aa, human
GQTITTPL

SEQ ID NO: 36 - protein, 8 aa, human
GQTLTTPL

SEQ ID NO: 37 - protein, 8 aa, human
GQIFSRSA

SEQ ID NO: 38 - protein, 8 aa, human
GQIHGLSP

SEQ ID NO: 39 - protein, 8 aa, human
GARASVLS

SEQ ID NO: 40 - protein, 8 aa, human
GCTLSAEE

SEQ ID NO: 41 - protein, 8 aa, human
GQNLSTSN

SEQ ID NO: 42 - protein, 8 aa, human
GAALTILV

SEQ ID NO: 43 - protein, 8 aa, human
GAALTLLG

SEQ ID NO: 44 - protein, 8 aa, human
GAQVSSQK

SEQ ID NO: 45 - protein, 8 aa, human
GAQLSRNT

SEQ ID NO: 46 - protein, 8 aa, human
GNAAAAKK

SEQ ID NO: 47 - protein, 8 aa, human
GNEASYPL

SEQ ID NO: 48 - protein, 8 aa, human
GSSKSKPK

SEQ ID NO: 49 - DNA, 38 bp, human
ccctctagacatatgtgaattccaccatggcccaggtc

SEQ ID NO: 50 - DNA, 25 bp, human
tgmggagacggtgaccrwggtccct

SEQ ID NO: 51 - DNA, 837 bp, human
  CDS (1)...(837)
  UNSURE (169): UNSURE
atggaacatctgtggttcttccttctcctggtggcagctcccagatgggtcctgtcccaggtgcaactgcagcagtcaggggctgagctggcaagacctggggcttcagtgaagttgtcctgcaaggcttctggctacacctttactagtcactggatgcagtgggtgagacagaggcctggacagggtctggaatggattgggactatttatcctggagatggtgatactaggtacactcagaatttcaagggcaaggccacattgactgcagataagtcctccaccacagcctacttacacctcagcagcttgtcatctgaagactctgcggtctattattgtgcaagagatgagattactacggttgtaccccgggggtttgcttactggggccaagggacctcggtcaccgtctcctcaggtggcggtggctcgggcggtggtggctcgggtggcggcggatctgagctcgtgctcacccaaaccccaacctccctggctncctctctgggagacagagtcaccatcagttgcagggcaagtcaggacattagcagttatttaaactggtatcagcagaaaccagatggaactattaaactcctgatctactacacatcaagattatattcaggagtcccaccaaggttcagtggcagtggggctggaacagattattctctcaccatcagcaacctggagcaagaagattttgccacttacttttgccaacagggtaatgtgattccgtacacgttcggaggggggaccaagctggaaatgaaacgggctgatgctgcaccaactgtaagcgaaaaggacgagctgtaataa

SEQ ID NO: 52 - protein, 277 aa, human
  UNSURE (169): UNSURE
MEHLWFFLLLVAAPRWVLSQVQLQQSGAELARPGASVKLSCKASGYTFTSHWMQWVRQRPGQGLEWIGTIYPGDGDTRYTQNFKGKATLTADKSSTTAYLHLSSLSSEDSAVYYCARDEITTVVPRGFAYWGQGTSVTVSSGGGGSGGGGSGGGGSELVLTQTPTSLAXSLGDRVTISCRASQDISSYLNWYQQKPDGTIKLLIYYTSRLYSGVPPRFSGSGAGTDYSLTISNLEQEDFATYFCQQGNVIPYTFGGGTKLEMKRADAAPTVSEKDEL

SEQ ID NO: 53 - DNA, 837 bp, human
  CDS (1)...(837)
atggaacatctgtggttcttccttctcctggtggcagctcccagatgggtcctgtcccaggtgcaactgcagcagtctggggctgagctgacaagacctggggcttcagtgaagttgtcctgcaaggcttctggctacacctttactagtcactggatgcagtgggtgagacagaggcctggacagggtctggaatggattgggactatttatcctggagatggtgatactaggtacactcagaatttcaagggcaaggccacattgactgcagataagtcctccaccacagcctacttacacctcagcagcttgtcatctgaagactctgcggtctattattgtgcaagagatgagattactacggttgtaccccgggggtttgcttactggggccaagggaccttggtcaccgtctcctcaggtggcggtggctcgggcggtggtgggtcgggtggcggcggatctgagctcgtgctcacccagtctccatccagtctgtctgcatcccttggagacacaattaccatcacttgccatgccagtcagaacattaatgtttggttaagttggtaccagcagaaaccaggaaatattcctcaactattgatctataaggcttccaacttgcacacaggcgtcccatcaaggtttagtggccgtggatctggaacaggtttcacattaaccatcagcagcctgcagcctgaagacattggcacttactactgtcaacagggtcaaagttatcctctgacgttcggtggaggcaccaagctggaaatcaaacgggctgatgctgcaccaactgtaagcgaaaaggacgagctgtaataa

SEQ ID NO: 54 - protein, 277 aa, human
MEHLWFFLLLVAAPRWVLSQVQLQQSGAELTRPGASVKLSCKASGYTFTSHWMQWVRQRPGQGLEWIGTIYPGDGDTRYTQNFKGKATLTADKSSTTAYLHLSSLSSEDSAVYYCARDEITTVVPRGFAYWGQGTLVTVSSGGGGSGGGGSGGGGSELVLTQSPSSLSASLGDTITITCHASQNINVWLSWYQQKPGNIPQLLIYKASNLHTGVPSRFSGRGSGTGFTLTISSLQPEDIGTYYCQQGQSYPLTFGGGTKLEIKRADAAPTVSEKDEL

SEQ ID NO: 55 - protein, 4 aa, human
RKKR

SEQ ID NO: 56 - protein, 8 aa, human
SIINFEKL

(19) United States
(12) Patent Application Publication
(10) Pub. No.: US 2006/0034834 A1
(43) Pub. Date: Feb. 16, 2006

(54) Intrabody-mediated control of immune reactions

(75) Inventors: Wayne A. Marasco, Wellesley, MA (US); Abner Mhashilkar, Cleveland, OH (US)

Correspondence Address: RONALD I. EISENSTEIN;NIXON PEABODY LLP, 100 summer street, BOSTON, MA, 02110, US

(73) Assignee: Dana-Farber Cancer Institute, Inc., Boston, MA (US)

(21) Appl. No.: 11/126,817

(22) Filed: May 11, 2005

Related U.S. Application Data
(62) Division of application No. 09/522,727, filed on Mar. 10, 2000.
Application No. 09/522,727 is a continuation of application No. PCT/US98/19563, filed on Sep. 18, 1998.
(60) Provisional application No. 60/059,339, filed on Sep. 19, 1997.

(51) Int. Cl. A61K 39/395; A61K 48/00

(52) U.S. Cl. 424/133.1; 514/44

(57) ABSTRACT

The present invention is directed to methods of altering the regulation of the immune system, e.g., by selectively targeting individual or classes of immunomodulatory receptor molecules (IRMs) on cells comprising transducing the cells with an intracellularly expressed antibody, or intrabody, against the IRMs. In a preferred embodiment the intrabody comprises a single chain antibody against an IRM, e.g, MHC-1 molecules.

### CROSS-REFERENCE TO RELATED APPLICATION

This application is a divisional application of co-pending application Ser. No. 09/522,727, filed Mar. 10, 2000, which was a U.S. National 371 entry of International Application PCT/GB2000/001415 filed 13 Apr. 2000, which claims benefit under 35 U.S.C. §119 of GB 9909349.4 filed 23 Apr. 1999. The specification of co-pending application Ser. No. 09/522,727 is hereby incorporated herein by reference.

### FIELD OF INVENTION

The present invention relates to the manipulation of immune responses in cells by targeting the cells with intrabodies.

### BACKGROUND OF THE INVENTION

Antigen presenting cells allow the immune system to monitor tissues for the presence of viral infections or tumors. In this process, proteins in the cytosol are hydrolyzed by proteosomes or by other proteinases, and some of the oligopeptide products are transferred into the endoplasmic reticulum (ER) by the transporter associated with antigen processing (TAP) and, after binding to newly assembled immunomodulatory receptor molecules (IMR), are transported to the plasma membrane. Since almost all proteins that are resident in the cytosol and ER are synthesized by the antigen presenting cells (APCs), this pathway provides a sampling of the peptides to the immune system. In most cases, these peptides are derived from autologous proteins and are ignored by the immune system due to self-tolerance. However, if cells display foreign peptides (viral or mutated gene products), the cytotoxic T-lymphocytes (CTLs) will kill the offending cells (Rock, K. L., Immunology Today 17:131-137 (1996).

Immunomodulatory receptor molecules (IRM) function to control and trigger immune responses by presenting pieces of the degraded proteins to the immune system. This is a tightly regulated system which typically helps protect the body from undesired intrusions of foreign matter such as viral infections and foreign cells. One example of an IRM is the major histocompatibility complex (MHC) molecule. The MHC molecules include 2 classes, class I and class II molecules. The classical major histocompatibility complex (MHC) class I pathway is operative in almost all cells. Functional class I molecules are found at the cell surface and comprise a tightly folded complex of class I chain glycoproteins and B₂-microglobulin and a short peptide derived from degradative turnover of intracellular proteins. MHC molecules are found on a wide variety of cell types and are efficiently internalized by endocytosis in numerous cell types. Signals of cellular distress are raised either when class I molecules contain foreign peptides of parasitic, bacterial or viral or tumor origin, which activate CTL, or when cell surface levels of class I drop to the point where NK cells are no longer inhibited [Parham, P., TIBS 21:427-433 (1996)]. Antigens seen by T cells are degraded inside a host cell before they are presented to the T cell on the surface of the host cell. The fragments of viral proteins wind up on the surface of the infected cell by associating with MHC molecules either on the surface of the cells or perhaps inside the cell. See e.g., Alberts, et al., Molecular Biology of the Cell, 2^{nd} ed. (1986), p. 1043.

Class I MHC pathway continuously shuttles peptides back and forth from the endoplasmic reticulum (ER) to the plasma membrane at the surface of the cell. The MHC peptide complex can bind to the T-cell receptor complex which in turn leads to activation of the T-cell.

Other examples of IRMs includes the numerous ligands and receptors involved in immune responses, for example, cytokines such as various interleukins, and co-stimulatory molecules such as B7-1 and B7-2. These molecules help to stimulate and/or enhance cellular immune reactions. For example, B7-1 and B7-2 interact with the cellular receptors CD 28 and CTLA-4 to turn on their activity and turn off their activity, respectively.

Other receptors are involved in activating T and B cells, such as CD40, CD 20 and CD 43. In this manner, IRMs play a very critical role in immunosurveillance against infectious agents and tumors. There are times when this tight regulation produces an undesired effect. This can be seen where one wants to add a foreign object to the body, for example, in organ transplantation or when vectors are being therapeutically added. For example, transplantation reactions, e.g., tissue rejection, are regulated by MHC class I molecules. Transplantation reactions include both the rejection of transplanted tissue by the recipient, as well as the rejection of recipient tissue by the graft. The latter process can occur in patients who receive bone marrow grafts as treatment for an immunodeficiency, i.e., it is a graft-versus-host response. Both types of reactions are directed against foreign cell-surface antigens called histocompatibility antigens. The most common of which are antigens encoded by genes for the major histocompatibility complex (MHC). It would therefore be useful to be able to selectively target IRMs, e.g., MHC molecules, such as MHC-1, or their pathways or sometimes even their targets to suppress or downregulate them in order to either prevent or minimize a transplantation reaction. However, it is also important that other cells maintain their ability to function. Thus, the method of selection should as specifically as possible target the IRMs of interest and not other molecules, e.g., receptors, etc., in the cell.

There are instances where a vector is used to deliver a desired DNA segment in order to express an antigen to obtain a desired immune reaction. Unfortunately, sometimes the vector itself generates an immune reaction that masks the immune reaction caused by the antigen. It would be desirable to selectively inhibit the reaction to the vector but not the desired response to the antigen.

IRMs are also involved in autoimmune reactions where the tolerance to self antigens has broken down, leading to various diseases. In these diseases, T and/or B cells act against their own tissue antigens. Again, MHC molecules, particularly MHC-1 molecules, have an active role in these reactions. Thus, it would be useful to be able to down regulate IRMs for the treatment of certain autoimmune diseases.

Finally, it would also be useful for gene therapy to be able to help regulate these molecules to decrease or prevent surface expression of certain IRMs on transduced cells to increase the time period for in-vivo survival of these cells. Such cells would avoid the immune responses of CTLs and NK cells. These cells can also be used as carriers of vaccines and other therapeutic molecules in-vivo.

Accordingly, it would be desirable to have a method of selectively targeting the IRM of interest, its pathway, or targets in order to regulate the system in a desired manner, such as to down regulate or inhibit the surface expression of IRMs.

### SUMMARY OF THE INVENTION

The present invention is directed to methods of altering the regulation of the immune system, e.g., by selectively targeting individual or classes of immunomodulatory receptor molecules (IRMs) on cells comprising transducing the cells with an intracellularly expressed antibody, or intrabody, against the IRMs. In preferred methods, one can target an epitope present on a number of IRMs, for example, MHC-1 molecules. In other instances one targets MHC class I molecules, MHC class II molecules, CD28 or CD40, T cell receptors, LMP2 molecules, LMP7 molecules and CD1 molecules.

The present invention is also directed to methods of selectively targeting components in the antigen processing pathways, instead of the IRM itself. For example, by blocking even one of these components, the immune response resulting from antigen presentation, can be regulated. For example, to modulate the MHC class I pathway, intrabodies can be used to target components in the pathway comprising MHC-1α chains, β2-microglobulin, TAP.1 molecules, TAP.2 molecules, calnexin, calreticulin and tapasin. Components of other pathways, e.g., MHC class II pathway, CD1 pathway, can also be selectively targeted by specific intrabodies in an analogous method.

The present invention is also directed to methods of selectively preventing presentation of an antigen on the cell comprising targeting the antigens or specific portion thereof that elicits the undesired immune response with an intrabody.

The intrabody comprises whole antibodies, heavy chains, Fab′ fragments, single-chain antibodies and diabodies. In one preferred method of the present invention, the intrabody comprises a single-chain antibody (sFv). If the target is a receptor, the antibody contains a leader sequence and an ER or Golgi appropriate retention signal, such as KDEL (SEQ ID NO: 17). Preferably, cells are transduced with a single-chain antibody to human MHC-1 (sFvMHC-1) containing a leader sequence and an endoplasmic reticulum (ER) such as, e.g., a KDEL (SEQ ID NO: 17) sequence or golgi apparatus retention signal. Such a method prevents expression of the MHC-1 molecules on the surface of cells. The downregulation of MHC-1 molecules is useful for controlling particular immune responses, such as tissue rejection, autoimmune diseases and bone marrow transplantation. In another embodiment, the target would be elsewhere in the cell and a functional leader sequence would not be present.

### BRIEF DESCRIPTION OF THE DRAWINGS

FIG. 1 shows a schematic illustration of MHC-1 surface expression, with the illustration on the left showing a normal pathway of MHC-1 cell surface expression, and the illustration on the right showing the cell surface expression in the presence of ER-expressed sFvhMHC-1.

FIGS. 2A through 2D show the sequences of certain single chain antibodies. FIGS. 2A and 2B show the primary nucleotide (SEQ ID NO: 51) and amino-acid (SEQ ID NO: 52) sequences of sFvMHC-1-5k and FIGS. 2C and 2D show the primary nucleotide (SEQ ID NO: 53) and amino acid (SEQ ID NO: 54) sequences in sFvMHC-1-8k (B).

FIG. 3 shows transient expression of sFvMHC-1 in COS-1 cells.

FIG. 4 shows the stable expression of sFvMHC-1 in Jurkat cells.

FIGS. 5A and 5B show the FACS analysis of Jurkat stable subclones.

FIG. 6 shows the FACS analysis of selected Jurkat stable subclones.

FIG. 7 shows the FACS analysis of one pRc/CMV empty vector and two sFvhMHC-1 subclones.

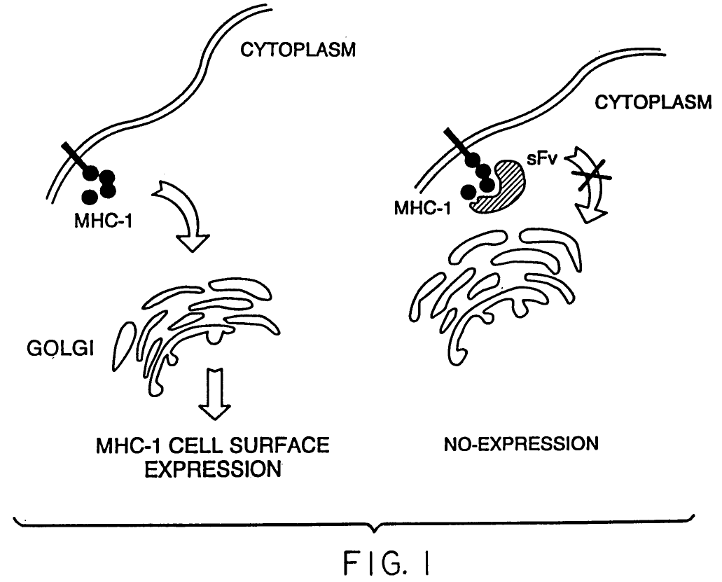

### DETAILED DESCRIPTION OF THE INVENTION

We have discovered methods of selective targeting of immunomodulatory receptor molecules (“IRMs”), their pathways or compounds that interact with such molecules which can be used to selectively regulate the immune system by controlling expression of these molecules on the surface of cells. More specifically, this method involves the use of intracellular binding to a desired target by an antibody. This method of intracellular antibody binding has been described in PCT/US93/06735, filed on Jan. 17, 1992 and U.S. patent application Ser. No. 08/350,215, filed on Dec. 6, 1994, which are incorporated herein by reference. The intracellularly expressed antibodies are referred to as intrabodies. Whole antibodies, heavy chains, Fab′ fragments, single chain antibodies and diabodies can be used. Preferably the intrabody is a single chain antibody, diabody, or Fab′. More preferably, it is a single chain antibody. For example, by using single-chain antibodies (intrabodies) to immunomodulatory receptor molecules, e.g., MHC class I molecules, surface expression of those IRMs is downregulated or inhibited.

The concept of “Intracellular Immunization” or “Intracellular Inhibition” has in the last decade emerged as an important strategy to counteract functionalities of pathogenic bacteria, viruses and parasites. Intracellular Immunization utilizes molecular modulators such as anti-sense RNA, ribozymes, dominant negative mutants and intracellular antibodies (intrabodies) for inhibiting functional gene expression within the cell. Previous studies have shown the efficacy of intrabodies (e.g., sFvs and Fabs) targeting expression in different compartments of the cell, including the nucleus, ER, cytoplasm, golgi, plasma membrane, mitochondria, where they act to counteract antigens or molecules in a specific pathway. [Marasco, W. A., et al, Proc. Natl. Acad. Sci., USA 90:7889-7893 (1993); Chen, S. Y., et al., Human Gene Therapy 5:595-601 (1994); Chen, S. Y., et al., Proc Natl Acad Sci, USA 91:5932-5936 (1994); Mhashilkar, A. M., et al., Embo J 14:1542-1551 (1995); Marasco, W. A., et al. Gene Therapy 4:11-15 (1997); Richardson, J. H., et al., Proc Natl Acad Sci, USA 92:3137-3141 (1995); Duan, L., et al., Human Gene Therapy 5:1315-1324 (1994)]. The antibodies can be localized to specific cellular compartments, e.g., the ER, nucleus, inner surface of the plasma membrane, the cytoplasm and the mitochondria. (See e.g., Marasco et al, 1993; Mhashilkar et al., 1995; Biocca et al., 1995).

The present invention uses the intrabodies to change the native immunoregulation, e.g., to inhibit transport of immunomodulatory molecules to the plasma membrane, and thereby decrease or prevent an immune response. Alternatively, the present invention uses intrabodies to intracellularly target an antigen such as a processed peptide before it interacts with the receptor protein. The methods of the present invention are useful in preventing tissue rejection, autoimmune diseases, etc.

The methods of the present invention enable the selective blockage of target antigens, such as surface expression of particular IRMs of interest. For example, it is known that there are different haplotypes of MHC class I molecules based on different protein chains. We have found that intrabodies can be designed to selectively target particular MHC class I molecules or alternatively, to target multiple class I molecules. This is accomplished by the choice of the epitope that the intrabody binds to. For example, by using a conserved epitope to generate the antibody multiple molecules can be knocked out by a single intrabody. Conversely, using an epitope unique to a particular molecule results in selective binding. The type of antibody can be generated readily by standard means based upon the particular objective. For example, the structure of most of these molecules and peptides are known, as are the conserved and unique regions of these molecules. Accordingly, by targeting any of these regions, the ultimate expression of the MHC molecules is prevented on the surface of the cells. This is particularly useful for targeting specific class I molecules that are known to be involved in particular immune responses, such as tissue rejection, autoimmune diseases or bone marrow transplantation.

The methods of the present invention are also useful for targeting IRMs in order to treat other diseases which have not traditionally been referred to as immune related diseases. For example, it has recently been shown that HLA-2 receptors have an association with early onset of Alzheimer's Disease. Thus, these molecules have been targeted with anti-inflammatory agents to treat people at risk for Alzheimer's disease. However, such agents can pose health problems that the present method does not.

The methods of the present invention can also be used to specifically target other molecules, e.g., the HLA-2 molecules or CD28 molecules and prevent their expression, while leaving other surface molecules unaffected.

In another preferred method of the present invention, the intrabodies are used to knockout multiple locuses of IRMs. That is, as briefly mentioned above, the intrabodies can be used to silence more than one single IRM in a family of proteins. For example, even though there are numerous haplotypes of MHC class I molecules, the α3 domain of HLA-A, HLA-B and HLA-C is conserved. Such a domain is sometimes referred to as monomorphic. By targeting a monomorphic region, a variety of molecules are targeted. Intrabodies of the present invention can be designed to be directed against an epitope on that alpha chain that is common to HLA-A, HLA-B and HIA-C. By doing so, one can effectively block the expression of multiple MHC molecules. Alternatively, by targeting unique polymorphic epitopes, only specific MHC molecules will be blocked. The choice depends upon the particular goal.

As discussed briefly above, the pathways that involve IRMs involve numerous components. Any component in the pathways which involve the IRMs, e.g., presentation of antigens, in the cell can be targeted by the methods of the present invention in order to modulate the immune response of that cell. For example, the MHC-I pathway is an elegant pathway that involves numerous molecules to ensure that the peptide becomes associated with the MHC-I molecule and then that the MHC-I-peptide complex is presented on the surface of the cell. In the first step of antigen presentation, the peptides that bind the MHC-I molecules are generated by proteasome-mediated cleavage of cytosolic proteins. These peptides are translocated into the ER by the transporter associated with antigen processing (TAP). TAP is a member of the ATP-binding cassette family of transporters and is composed of two homologous MHC-encoded subunits, TAP.1 and TAP.2. Assembly of the MHC class I-peptide complex is initiated in the ER by formation of MHC class I-β2-microglobulin dimers and involves the molecules calnexin and calreticulin. See e.g., Ortmann, B. et al., Science, Vol. 277, 1306-1309 (Aug. 29, 1997). Before the peptide binds to MHC-1, calreticulin-associated class I molecules bind to TAP. This interaction is mediated by a molecule called tapasin. Id. After TAP translocates an allele-specific class I binding peptide, the class I molecule dissociates from the TAP complex. Id. The peptide bound newly assembled MHC-I molecules are then transported by an exocytic pathway to the plasma membrane. The peptides are thus presented to CD8+ cytotoxic T lymphocytes (CTLs) bearing the appropriate T-cell receptor (TCR). See e.g., Rock, K. L., Immunology Today, Vol. 17, No. 3, 131-137 (March 1996); Rammensee, H., et al., Immunognetics, Vol. 41, 178-228 (1995).

Any of these components of the MHC pathway, e.g., the α chains of the MHC, β2 microglobulin molecules, calnexin and calreticulin, TAP, including TAP.1 and TAP.2, and tapasin, even the enzymes that degrade the peptide in the proteasome, or even the particular peptide, can be targeted by intrabodies, as described herein, in order to modulate the immune response of particular cells of interest. Any intrabody prepared must be targeted to the particular compartment in which the component is localized. For example, to target the ER components of MHC synthesis, the intrabodies must be directed to the ER and contain an appropriate leader sequence as further described below.

For example, TAP is necessary for efficient peptide transport into the ER. TAP is a heterodimer, where each subunit has an ATP-binding domain. Both these subunits are required for peptide transport. ATP hydrolysis is also required for translocation of peptide into the ER. See e.g., Hill, A. and Ploegh, H., Proc. Natl. Acad. Sci., Vol. 92, pp. 341-343 (January 1995). Thus, an intrabody against one or both of the TAP subunits would prevent assembly of the TAP molecule and effectively block transport of the antigenic peptide into the ER. This would prevent association of the antigen with the MHC molecule and in the end, prevent presentation of the antigen on the surface of the cell. Alternatively, an intrabody can be designed to target the antigen binding site on the assembled TAP molecule. In yet another embodiment, an intrabody can be used to target the TAP ATP-binding site to prevent translocation of the peptide into the ER.

In other embodiments, the assembly of the MHC molecules themselves can be prevented by specifically targeting a component in the MHC assembly line. In this case, the interaction between the newly synthesized MHC class I heavy chains, β2-microglobulin, calnexin and calreticulin can be inhibited by targeting any one or a mixture of these components. For example, an intrabody to calnexin can be prepared according to the present teachings, and containing an ER specific leader sequence in order to prevent the interaction of calnexin with the MHC subunits.

Similarly, in order to prevent the binding of MHC molecules to TAP, the interaction with tapasin can be prevented by targeting that molecule with an tapasin-specific intrabody. This molecule has recently been sequenced. Ortmann, B., et al., Science, Vol. 277, 1306-1309 (Aug. 29, 1997).

As mentioned above, the first step of the antigen presenting pathway involves the cytosolic degradation of molecules, such as proteins. Degradation typically involves covalent conjugation of the protein to multiple molecules of the polypeptide ubiquitin. This process marks the protein for hydrolysis by the 26S proteasome. See e.g., Goldberg, A. L., Science, Vol. 268, 522-523 (Apr. 28, 1995). Two subunits of the proteasome (LMP2 and LMP7) involved in the MHC-1 pathway are encoded in the MHC locus. See e.g., Rock, K. L., et al., Cell, Vol. 78, 761-771 (Sep. 9, 1994) (see articles cited therein).

The methods of the present invention can be used to target the components of this first stage in antigen presentation. For example, intrabodies to ubiquitin can be used to prevent conjugation of the antigenic protein to ubiquitin, in order to prevent the interaction with the proteasome. Similarly, intrabodies can be used to target one or both of the two subunits of the proteasome, LMP2 and LMP7, to prevent assembly of the proteasome. These are examples of some of the numerous targets available to prevent peptide production from cell protein degradation and in turn block assembly of MHC-1 molecules by using the methods of the present invention. (See e.g. Rock, K. L., supra)

Similarly, intrabodies of the present invention directed to different types of molecules, e.g., different MHC class I molecules, can be mixed in a cocktail to selectively target multiple loci on the cells. This “cocktail” approach (i.e. mixture of antibodies) can be used to silence the proteins of interest, whether they be receptor proteins, viral proteins, or other antigens. The use of a cocktail of antibodies enables the targeting of a variety of proteins at one time. This is useful to knock out a range of receptors, or to make it more difficult for mutants to evolve which will produce functional target protein capable of avoiding the antibody. For example, a cocktail of antibodies to unconserved regions of the various haplotypes of MHC-1 molecules can be used to knock out multiple loci. Such “cocktails” can be administered together or by co-transfections. It is preferred that no more than about three proteins in the same intracellular region are targeted, preferably no more than about two, for example, targeting CD28 and HLA1A at the endoplasmic reticulum. As long as another intracellular target is in a different cellular region, i.e. nucleus versus endoplasmic reticulum, it can also be targeted without having a detrimental effect on antibody production.

Another preferred cocktail would be of antibodies to the same target, but at various intracellular locations. This could be done using different localization sequences. Thus, if some target is not bound to the antibody at one location and, for instance, is further processed, it can be targeted at a subsequent location. For example, with a target MHC-1 receptor one could use localization sequences to target the protein or components of the system at a number of points in its processing path. For example, using one antibody to target the β-microglobulin and a second antibody to target the α chain of the MHC-1 receptor.

Other IRMs of interest include CD1 proteins, which are related in some ways to MHC molecules. CD1 molecules are not polymorphic, like MHC molecules. However, they are remotely homologous to MHC in their α1 and α2 domains. CD1 molecules are expressed in the thymus, on antigen-presenting dendritic cells in different tissues and on cytokine-activated monocytes. Sieling, P. A., et al., Science, Vol. 269, 227-230 (Jul. 14, 1995). CD1 molecules comprise different isotypes (CD1a, b, c, d, and e) that are conserved in several mammalian species. Bendelec, A., Science, Vol. 269, pp. 185-186 (Jul. 14, 1995). It has been found that isotype CD1b presents lipids, such as lipoglycans, rather than peptides, to T cells. Bendelec, A. supra; Sieling, P. A., et al., Science, Vol. 269, 227-230 (Jul. 14, 1995); Beckman, E. M., et al., Nature, Vol. 372, 691-694 (Dec. 15, 1994). None of the MHC-encoded antigen processing molecules, e.g., TAP, is required for lipid presentation. Thus, other molecules that are involved would be used for CD1 trafficking and lipid antigen processing. Bendelec, A., supra. A peptide binding motif has been found through screening random peptide phage display libraries with soluble empty mouse CD1 (mCD1). Castano, A. R., et al., Science, Vol. 269, p.223-226 (Jul. 14, 1995). CD1d, the only isotype expressed by mouse and rat, should specifically bind peptides. Bendelac, A., supra.

In one embodiment of the present invention, intrabodies target CD1 molecules in order to prevent expression of CD1 molecules on the surface of cells. As discussed above, with respect to MHC-1 molecules, the IRMs can be targeted a number of different ways. For example, the conserved regions of the isotypes can be targeted to knock out the whole range of CD1 molecules. Alternatively, the unique regions of a particular isotype can be targeted to knock out one particular isotype.

In another example, the CD1 antigen presenting pathway can be modulated. Intrabodies to the components of this pathway can be targeted in order to prevent antigen presentation, e.g. by preventing assembly of the CD1 molecule, binding of the antigen to the CD1 molecule or transport of the CD1 antigen complex to the surface of the cell.

In yet another embodiment, MHC class II molecules and its AP pathway, as well as its synthetic pathway, can be targeted using the methods of the present invention. MHC class II molecules acquire antigenic peptides in the endosomal/lysosomal compartments of the cell. Teyton, L., et al., The New Biologist, Vol. 4, No. 5, 441-447 (1992). The MHC class II molecule is composed of 2 non-identical glycoproteins, the ∀ and ∃ chains. A second membrane glycoprotein, the invariant chain (Ii), complexes with the ∀ and ∃ chains in the ER to stabilize the MHC-II in the absence of a bound peptide. Ii also guides the MHC-II to the endocytic pathway. Ghosh, P. et al., Nature, Vol. 378, p. 457-462 (November 1995); Tulp, A., et al., Nature, Vol. 369, 120-126 (May, 1994). It is removed by proteolysis in the endosome before the antigenic peptide is loaded on the MHC-II molecule. A nested set of 20-24 residue Ii fragments (within residues 81-104) is called CLIP (class II associated invariant chain peptide). This CLIP segment has an important role in the functioning of Ii and MHC-II molecules. For example, studies have shown that CLIP is necessary for ∀∃ assembly in vivo. Id. CLIP must be removed from MHC-II molecules before peptide loading. This is believed to occur in the endosomal/lysosomal compartment. The invariant chain is then degraded. Ghosh, P. supra.

Other IRMs of interest include MHC class II molecules, CD28 molecules and CD40 molecules CD-1 molecules. MHC class II molecules are involved in MHC class II molecules are located primarily on cells involved in immune responses and are recognized by helper T cells, which interact with cells involved in immune responses, e.g., B cells and antigen presenting cells (APC). Activation of helper T cells is required in order to stimulate the response of other lymphocytes to antigens. Activation occurs when a helper T cell recognizes an antigen bound to an MHC class II molecule on an APC. The methods of the present invention are useful in mediating MHC class II molecules and regulating the activity of helper T cells. CD28 and B7 receptors are co-stimulatory molecules which trigger co-stimulatory signals for optimal T cell activation. CD40 is a receptor which activates a number of effects in B cells. Intrabodies to these receptors can be produced and used according to the methods of the present invention to specifically target and control the surface expression of these receptors.

The components of the MHC-II pathway can be targeted using intrabodies as described herein. For example, intrabodies directed to the ER specific for the ∀ chain or ∃ chain would prevent assembly of the MHC-II molecules. In another embodiment, an intrabody could be designed to bind to the ∀∃ complex where CLIP normally binds, i.e., homologous to CLIP. Such an intrabody should prevent the binding of antigenic peptides to the MHC-II molecules.

In yet another embodiment of the present invention, the intrabodies of the present invention can be used to knock out the immune response in a particular tissue or portion of the body to prepare it for cell or tissue transplantation. In such an embodiment, a constitutive vector is used to transduce the target cells in the area of interest, e.g., in an arthritic joint, the pleural cavity or central nervous system. The intrabodies are introduced into the cells and prevent expression of the IRMs of interest in the host cells while the vector continues to produce the intrabodies. After transplantation occurs, the host cells will not reject the transplanted tissue. After a particular amount of time, the vector no longer produces the intrabodies and the host cells slowly begin to express the IRM but accommodation should occur, consequently, the cells return to their normal functioning and accommodate the transplanted cells or tissue. Alternatively, an organ or tissue for transplantation can be perfused ex vivo with the intrabody of interest. For example, a kidney is perfused prior to implantation, in order to precondition the cells with the desired vector. Similarly, ∃ islet cells can be transduced with the intrabody of interest and injected into the pancreas.

In many cases, it is desirable to knock out the antigen itself, before it binds the IRM, e.g., MHC-1 molecules, to prevent presentation on the cell surface. In such a case, intrabodies to the antigen, be it a peptide, or its degradation product, can be used to selectively prevent the binding of antigen to the IRM. The intrabody can be targeted to the different cellular compartments, by using the appropriate leader sequence, to intercept the antigen at various points along the antigen presentation pathway. For example, SIINFEKL (SEQ ID NO: 56) is a known cellular degradation product of ovalbumin. It is known that introduction of ovalbumin into the cytosol leads to its proteolytic processing and presentation on MCH-1 molecules. Moore et al., Cell, Vol. 54, 777-785 (1988); Rock, et al., Cell, Vol. 78, 761-771 (1994). An antigen such as albumin could be targeted in the cytosol before degradation by the proteasome. After degradation, one could target the degradation product, e.g., SIINFEKL, (SEQ ID NO: 56) prior to binding with TAP, or in the ER, prior to binding the MHC-1 molecule. The binding of the intrabody to the antigen prevents presentation of the antigen on the cell surface.

Similarly, as discussed above, vectors are useful to deliver a desired DNA segment to particular cells in order to express an antigen which then invokes a desired immune response. However, in some instances, the vector itself generates an immune reaction that masks the desired immune reaction. In such reactions, the vector is degraded and the viral peptides are presented to T cells via MHC-1 molecules on the surface of the infected cells. This invokes an immune reaction to the viral peptides/antigens which interfere with the desired reaction. In another embodiment of the present invention, intrabodies are used to interact with the interfering viral peptides within the cell to block the transport of these peptides to the surface of the cell. That is, the intrabodies inhibit the interaction of these peptides with the MHC-1 molecules, preventing the presentation of these antigens on the cell surface and preventing the undesired immune response.

A wide range of approaches to transduce the cells can be used, including viral vectors, “naked” DNA, adjuvant assisted DNA, gene gun, catheters, etc. For example, retroviral vectors can also be used to transduce cells with intrabodies to IRMs on antigens of interest. For example, we have cloned sFvhMHC-1 in the Murine Maloney retroviral LN vector [Miller, A. D., Immunology vol. 158 (1994)]. This retroviral construct can be used to infect cells with the intrabodies to the IRM of interest. Other vector systems useful in practicing the present invention include the adenoviral and HIV-1 based vectors, such as pseudotyped HIV-1. sFvMHC-1 construction of these vectors enable the transduction of human hemopoietic and non-hemopoeitic cell lines.

Cells in which IRMs, e.g., MHC-1 molecules, or their pathways are downregulated or inhibited are also useful as carriers of vaccines and other therapeutic molecules, because the lack of immunomodulatory molecules on the surface of these cells may prolong the in vivo survival rate of these cells.

The antibodies for use in the present invention can be obtained by methods known in the art against the IRM or antigen of interest. For example, single chain antibodies are prepared according to the teaching of PCT/US93/06735, filed on Jan. 17, 1992 and U.S. patent application Ser. No. 08/350,215, filed on Dec. 6, 1994, incorporated herein by reference. In one embodiment, the antibody is constructed so that it is directed to and remains in the lumen of the ER of the target cell. Such construction can be readily achieved by known methods so that the intrabody contains an ER-retention signal, e.g., KDEL (SEQ ID NO:17). An example setting forth the construction of an ER-expressed intrabody to MHC-1 molecules using ATCC HB94 hybridoma cells (fusion name BB7.7, anti-HLA-A,B,C) is set forth below. Based on this teaching and the known art, intrabodies, e.g., sFvs, to other IRMs can readily be obtained by the skilled artisan.

The target molecules can be present in a wide range of hosts, including animals and plants. Preferably, the host is an animal and more preferably, the species is one that has industrial importance such as fowl, pigs, cattle, cows, sheep, etc. Most preferably, the species is a human.

As discussed above, in one preferred embodiment of the present invention, the intrabody is a single chain antibody (sFv) to the IRM or antigen of interest. Determination of the three-dimensional structures of antibody fragments by X-ray crystallography has lead to the realization that variable domains are each folded into a characteristic structure composed of nine strands of closely packed β-sheets. The structure is maintained despite sequence variation in the V_{H} and V_{L} domains [Depreval, C., et al., J. Mol. Biol. 102:657 (1976); Padlan, E A., Q. Rev. Biophys. 10:35 (1977)]. Analysis of antibody primary sequence data has established the existence of two classes of variable region sequences: hypervariable sequences and framework sequences [Kabat, E. A., et al., Sequences of Protein of Immunological Interests, 4th ed. U.S. Dept. Health and Human Services (1987)]. The framework sequences are responsible for the correct β-sheet folding of the V_{H} and V_{L} domains and for the interchain interactions that bring the domains together. Each variable domain contains three hypervariable sequences which appear as loops. The six hypervariable sequences of the variable region, three from the V_{H} and three from the V_{L} form the antigen binding site, and are referred to as a complementarity determining region (CDRs).

By cloning the variable region genes for both the V_{H} and V_{L} chains of interest, it is possible to express these proteins in bacteria and rapidly test their function. One method is by using hybridoma mRNA or splenic mRNA as a template for PCR amplification of such genes [Huse, et al., Science 246:1276 (1989)]. For example, intrabodies can be derived from murine monoclonal hybridomas [Richardson J. H., et al., Proc Natl Acad Sci USA Vol. 92:3137-3141 (1995); Biocca S., et al., Biochem and Biophys Res Comm, 197:422-427 (1993) Mhashilkar, A. M., et al., EMBO J. 14:1542-1551 (1995)]. These hybridomas provide a reliable source of well-characterized reagents for the construction of intrabodies and are particularly useful when their epitope reactivity and affinity has been previously characterized. Another source for intrabody construction includes the use of human monoclonal antibody producing cell lines. [Marasco, W. A., et al., Proc Natl Acad Sci USA, 90:7889-7893 (1993); Chen, S. Y., et al., Proc Natl Acad Sci USA 91:5932-5936 (1994)]. Another example includes the use of antibody phage display technology to construct new intrabodies against different epitopes on a target molecule. [Burton, D. R., et al., Proc Natl Acad Sci USA 88:10134-10137(1991); Hoogenboom H. R., et al., Immunol Rev 130:41-68 (1992); Winter G., et al., Annu Rev Immunol 12:433-455 (1994); Marks, J. D., et al., J Biol Chem 267: 16007-16010 (1992); Nissim, A., et al., EMBO J 13:692-698 (1994); Vaughan T. J., et al., Nature Bio 14:309-314 (1996); Marks C., et al., New Eng J Med 335:730-733 (1996)]. For example, very large naïve human sFv libraries have been and can be created to offer a large source or rearranged antibody genes against a plethora of target molecules. Smaller libraries can be constructed from individuals with autoimmune [Portolano S., et al., J Immunol 151:2839-2851 (1993); Barbas S. M., et al., Proc Natl Acad Sci USA 92:2529-2533 (1995)] or infectious diseases [Barbas C. F., et al., Proc Natl Acad Sci USA 89:9339-9343 (1992); Zebedee S. L., et al., Proc Natl Acad Sci USA 89:3175-3179 (1992)] in order to isolate disease specific antibodies.

Other sources of intrabodies include transgenic mice that contain a human immunoglobulin locus instead of the corresponding mouse locus as well as stable hybridomas that secrete human antigen-specific antibodies. [Lonberg, N., et al., Nature 368:856-859 (1994); Green, L. L., et al., Nat Genet 7:13-21 (1994)]. Such transgenic animals provide another source of human antibody genes through either conventional hybridoma technology or in combination with phage display technology. In vitro procedures to manipulate the affinity and fine specificity of the antigen binding site have been reported including repertoire cloning [Clackson, T., et al., Nature 352:624-628 (1991); Marks, J. D., et al., J Mol Biol 222:581-597 (1991); Griffiths, A. D., et al., EMBO J 12:725-734 (1993)], in vitro affinity maturation [Marks, J. D., et al., Biotech 10:779-783 (1992); Gram H., et al., Proc Natl Acad Sci USA 89:3576-3580 (1992)], semi-synthetic libraries [Hoogenboom, H. R., supra; Barbas, C. F., supra; Akamatsu, Y., et al., J Immunol 151:4631-4659 (1993)] and guided selection [Jespers, L. S., et al., Bio Tech 12:899-903 (1994)]. Starting materials for these recombinant DNA based strategies include RNA from mouse spleens [Clackson, T., supra] and human peripheral blood lymphocytes [Portolano, S., et al., supra; Barbas, C. F., et al., supra; Marks, J. D., et al., supra; Barbas, C. F., et al., Proc Natl Acad Sci USA 88: 7978-7982 (1991)] and lymphoid organs and bone marrow from HIV-1-infected donors [Burton, D. R., et al., supra; Barbas, C. F., et al., Proc Natl Acad Sci USA 89:9339-9343 (1992)].

Thus, one can readily screen an antibody to insure that it has a sufficient binding affinity for the antigen of interest. The binding affinity (Kd) should be at least about 10⁻⁷ l/M, more preferably at least about 10⁻⁸ l/M.

The sFv sequences useful in the present invention will properly fold even under the reducing conditions sometimes encountered intracellularly. The sFv typically comprises a single peptide with the sequence V_{H}-linker-V_{L} or V_{L}-linker-V_{H} or a linkerless diabody. If a linker is used, it is chosen to permit the heavy chain and light chain to bind together in their proper conformational orientation. See for example, Huston, J. S., et al., Methods in Enzym. 203:46-121 (1991), which is incorporated herein by reference. Thus, the linker should be able to span the 3.5 nm distance between its points of fusion to the variable domains without distortion of the native Fv conformation. The amino acid residues constituting the linker must be such that it can span this distance and should be 5 amino acids or larger. The amino acids chosen also need to be selected so that the linker is hydrophilic so it does not get buried into the antibody. Preferably, the linker should be at least about 10 residues in length. Still more preferably it should be about 15 residues. While the linker should not be too short, it also should not be too long as that can result in steric interference with the combining site. Thus, it preferably should be 25 residues or less. The linker (Gly-Gly-Gly-Gly-Ser)₃ (SEQ ID NO:1) is a preferred linker that is widely applicable to many antibodies as it provides sufficient flexibility. Other linkers include Glu Ser Gly Arg Ser Gly Gly Gly Gly Ser Gly Gly Gly Gly Ser (SEQ ID NO:2), Glu Gly Lys Ser Ser Gly Ser Gly Ser Glu Ser Lys Ser Thr (SEQ ID NO:3), Glu Gly Lys Ser Ser Gly Ser Gly Ser Glu Ser Lys-Ser Thr Gln (SEQ ID NO:4), Glu Gly Lys Ser Ser Gly Ser Gly Ser Glu Ser Lys Val Asp (SEQ ID NO:5), Gly Ser Thr Ser Gly Ser Gly Lys Ser Ser Glu Gly Lys Gly (SEQ ID NO:6), Lys Glu Ser Gly Ser Val Ser Ser Glu Gln Leu Ala Gln Phe Arg Ser Leu Asp (SEQ ID NO:7), and Glu Ser Gly Ser Val Ser Ser Glu Glu Leu Ala Phe Arg Ser Leu Asp (SEQ ID NO:8). Alternatively, one can take a 15-mer, such as the (Gly-Gly-Gly-Gly-Ser)₃ (SEQ ID NO:1) linker, (although any sequence can be used) and randomize the amino acids in the linker through mutagenesis. Then the antibodies with the different linkers can be pulled out with phage display vectors and screened for the highest affinity single chain antibody generated.

Diabodies are dimeric antibodies fragments which are bispecific molecules. They are formed by cross-pairing two sFv molecules which each consist of a heavy chain variable domain (V_{H}) connected to a light chain variable domain (V_{L}) by either a shortened linker or no linker. The shortened/no linker prevents the domains on the same chain from pairing with each other. The two chains instead dimerize, forming a bivalent fragment. Bispecific fragments can be formed by the co-expression of two different chains, V_{H}A-V_{L}B and V_{H}B-V_{L}A, in the same cell. The diabody can be either monospecific or bispecific. McGuinness, B. T., et al., Nature Biotechnology, Vol. 14, 1149-1154 (September 1996); Hollinger, P., et al., Current Opinions in Biotechnol., Vol. 4, 446-449 (1993). Phage display libraries for diabodies have been described and can be used to generate thousands of different bispecific molecules and to select diabodies having the greatest binding affinity, epitope recognition and pairing. McGuinness, B. T., supra.

When the target is not in the ER or golgi apparatus, the gene does not encode a functional leader sequence for the variable chains, as it is preferable that the antibody does not encode a leader sequence. The nucleotides coding for such binding portion of the antibody preferably do not encode the antibody's secretory sequences (i.e. the sequences that cause the antibody to be secreted from the cell). Such sequences can be contained in the constant region. Preferably, one also does not use nucleotides encoding the entire constant region of the antibodies. More preferably, the gene encodes less than six amino acids of the constant region. However, when targeting an ER or golgi located target, a leader sequence will result in the antibody being brought to those compartments. Preferably an ER or golgi retention sequence is also present. This latter sequence is preferably added to the carboxy portion.

As discussed above, the immune system can be used to produce an antibody which will bind to a specific molecule such as a target protein by standard immunological techniques. For example, using the protein or an immunogenic fragment thereof or a peptide chemically synthesized based upon such protein or fragment. Any of these sequences can be conjugated, if desired, to keyhole limpet hemocyanin (KLH) and used to raise an antibody in animals such as a mice, rabbits, rats, and hamsters. Thereafter, the animals are sacrificed and their spleens are obtained. Monoclonal antibodies are produced by using standard fusion techniques for forming hybridoma cells. See, Kohler, G., et al. Nature 256:495 (1975). This typically involves fusing an antibody-producing cell (i.e., spleen) with an immortal cell line such as a myeloma cell to produce the hybrid cell.

Another method for preparing antibodies is by in vitro immunization techniques, such as using spleen cells, e.g., a culture of murine spleen cells, injecting an antigen, and then screening for an antibody produced to said antigen. With this method, as little as 0.1 micrograms of antigen can be used, although about 1 microgram/milliliter is preferred. For in vitro immunization, spleen cells are harvested, for example, mice spleen cells, and incubated at the desired amount, for example, 1×10⁷ cells/milliliter, in medium plus with the desired antigen at a concentration typically around 1 microgram/milliliter. Thereafter, one of several adjuvants depending upon the results of the filter immunoplaque assay are added to the cell culture. These adjuvants include N-acetylmuramyl-L-alanyl-D-isoglutamine [Boss, Methods in Enzymology 121:27-33 (1986)], Salmonella typhimurium mitogen [Technical Bulletin, Ribi ImmunoChem. Res. Inc., Hamilton, Montana] or T-cell condition which can be produced by conventional techniques [See, Borrebaeck, C. A. K., Mol. Immunol. 21:841-845 (1984); Borrebaeck, C. A. K., J. Immunol. 136:3710-3715 (1986)] or obtained commercially, for example, from Hannah Biologics, Inc. or Ribi ImmunoChem. Research Inc. The spleen cells are incubated with the antigen for four days and then harvested.

Single cell suspensions of the in vitro immunized mouse spleen cells are then incubated, for example on antigen-nitrocellulose membranes in microfilter plates, such as those available from Millipore Corp. The antibodies produced are detected by using a label for the antibodies such as horseradish peroxidase-labeled second antibody, such as rabbit anti-mouse IgA, IgG, and IgM. In determining the isotype of the secreted antibodies, biotinylated rabbit anti-mouse heavy chain specific antibodies, such as from Zymed Lab., Inc. can be used followed by a horseradish peroxidase-avidin reagent, such as that available from Vector Lab.

The insoluble products of the enzymatic reaction are visualized as blue plaques on the membrane. These plaques are counted, for example, by using 25 times magnification. Nitrocellulose membrane of the microfilter plaques readily absorb a variety of antigens and the filtration unit used for the washing step is preferred because it facilitates the plaque assay.

One then screens the antibodies by standard techniques to find antibodies of interest. Cultures containing the antibodies of interest are grown and induced and the supernatants passed through a filter, for example, a 0.45 micromiter filter and then through a column, for example, an antigen affinity column or an anti-tag peptide column. The binding affinity is tested using a mini gel filtration technique. See, for example, Niedel, J., Biol. Chem. 256:9295 (1981). One can also use a second assay such as a radioimmunoassay using magnetic beads coupled with, for example, anti-rabbit IgG to separate free ¹²⁵I-labeled antigen from ¹²⁵I-labeled antigen bound by rabbit anti-tag peptide antibody. In a preferred alternative one can measure “on” rates and “off” rates using, for example, a biosensor-based analytical system such as “BIAcore” from Pharmacia Biosensor AB [See, Nature 361:186-187 (1993)].

This latter technique is preferred over in vivo immunization because the in vivo method typically requires about 50 micrograms of antigen per mouse per injection and there are usually two boosts following primary immunization for the in vivo method.

Alternatively, one can use a known antibody to the target protein. Thereafter, a gene to at least the antigen binding portion of the antibody is synthesized as described below. As described briefly above, in some preferred embodiments it will also encode an intracellular localization sequence such as one for the endoplasmic reticulum, nucleus, nucleolar, etc. When expression in the ER normal antibody secretory system such as the endoplasmic reticulum-golgi apparatus is desired, a leader sequence should be used. To retain such antibodies at a specific place, a localization sequence such as the KDEL (SEQ ID NO: 17) sequence (ER retention signal) may be used. In some embodiments the antibody gene preferably also does not encode functional secretory sequences.

Antibody genes can be prepared based upon the present disclosure by using known techniques.

Using any of these antibodies, one can construct V_{H} and V_{L} genes. For instance, one can create V_{H} and V_{L} libraries from murine spleen cells that have been immunized either by the above-described in vitro immunization technique or by conventional in vivo immunization and from hybridoma cell lines that have already been produced or are commercially available. One can also use commercially available V_{H} and V_{L} libraries. One method involves using the spleen cells to obtain mRNA which is used to synthesize cDNA. Double stranded cDNA can be made by using PCR to amplify the variable region with a degenative N terminal V region primer and a J region primer or with V_{H} family specific primers, e.g., mouse-12, human-7.

For example, the genes of the V_{H} and V_{L} domains of the desired antibody such as one to MHC-1 molecules can be clone and sequenced. The first strand cDNA can be synthesized from, for example, total RNA by using oligo dT priming and the Moloney murine leukemia virus reverse transcriptase according to known procedures. This first strand cDNA is then used to perform PCR reactions. One would use typical PCR conditions, for example, 25 to 30 cycles using e.g. Vent polymerase to amplify the cDNA of the immunoglobulin genes. DNA sequence analysis is then performed. [Sanger, et al., Proc. Natl. Acad. Sci. USA 79:5463-5467 (1977)].

Both heavy chain primer pairs and light chain primer pairs can be produced by this methodology. One preferably inserts convenient restriction sites into the primers to make cloning easier.

As an example of the strategy that is used, heavy chain primer pairs consist of a forward V_{H} primer and a reverse J_{H} primer, each containing convenient restriction sites for cloning can be prepared. One could use, for example, the Kabat data base on immunoglobulins [Kabat, et al., supra] or Vbase database (I. Tomlinson (pub. by MRC); see also Tomlinson, I. M., et al., EMBO J., 14:4628-4638 (1995)) to analyze the amino acid and codon distribution found in the seven distinct human V_{H} families. From this, a 35 base pair universal 5′ V_{H} primer is designed. One could use a primer such as TTTGCGGCCGCTCAGGTGCA(G/A)CTGCTCGAGTC(T/C)GG (SEQ ID NO:9), which is degenerate for two different nucleotides at two positions and will anneal to the 5′ end of FR1 sequences. A restriction site such as the 5′ Not I site (left-underlined) can be introduced for cloning the amplified DNA and is located 5′ to the first codon to the V_{H} gene. Similarly, a second restriction site such as an internal XhoI site can be introduced as well (right-underlined).

Similarly, a 66-base pair J_{H} region oligonucleotide can be designed for reverse priming at the 3′ end of the heavy chain variable gene, e.g., AGATCCGCCGCCACCGCTCCCACCACCTCCGGAGCCACCGCCACCTGAGGTGACCGTGACC (A/G) (G/T) GGT (SEQ ID NO:10). This primer additionally contains a 45 nucleotide sequence that encodes a linker, such as the (Gly-Gly-Gly-Gly-Ser)₃ (SEQ ID NO:1) interchange linker. This primer contains two degenerate positions with two nucleotides at each position based on the nucleotide sequence of the six human J_{H} region minigenes. Restriction sites can be used, for example, a BspEI site (left-underlined) is introduced into the interchange linker for cohesive end ligation with the overlapping forward Vₖₐₚₚₐ primer. An internal BsTEII site (right-underlined) is introduced as well for further linker exchange procedures.

A similar strategy using the 45 nucleotide interchange linker is incorporated into the design of the 69 nucleotide human Vₖₐₚₚₐ primer. There are four families of human Vₖₐₚₚₐ genes. The 5′ Vₖₐₚₚₐ primer

GGTGGCGGTGGCTCCGGAGGTGGTGGGAGCGGTGGCGGCGGATCTGAGCTC(G/C)(T/A)G(A/C)TGACCCAGTCTCCA (SEQ ID NO:11), which will anneal to the 5′ end of the FR1 sequence is degenerate at 3 positions (2 nucleotides each). The interchange linker portion can contain a BspEI site for cohesive end cloning with the reverse J_{H} primer, other restriction sites can also be used. An internal SacI site (right-underlined) can be introduced as well to permit further linker exchange procedures.

The reverse 47 nucleotide Cₖₐₚₚₐ primer (Kabat positions 109-113) GGG TCTAGACTCGAGGATCCTTATTAACGCGTTGGTGCAGCCACAGT (SEQ ID NO:12) is designed to be complementary to the constant regions of kappa chains (Kabat positions 109-113). This primer will anneal to the 5′ most end of the kappa constant region. The primer contains an internal MluI site (right-underlined) proceeding two stop codons. In addition, multiple restriction sites such as Bam HI XhoI/XbaI (left-underlined) can be introduced after the tandem stop codons. A similar reverse nucleotide C-kappa primer such as a 59 nucleotide primer can also be designed that will contain a signal for a particular intracellular site, such as a carboxy terminal endoplasmic reticulum retention signal, Ser-Glu-Lys-Asp-Glu-Leu (SEQ ID NO:13) (SEKDEL),

GGGTCTAGACTCGAGGATCCTTATTACAGCTCGTCCTTTT

CGCTTGGTGCAGCCACAGT (SEQ ID NO:14). Similar multiple restriction sites (Bam HI XhoI/XbaI) can be introduced after the tandem stop codons.

After the primary nucleotide sequence is determined for both the heavy and kappa chain genes and the germ line genes are determined, a PCR primer can then be designed, based on the leader sequence of the V_{H} 71-4 germ line gene. For example, the V_{H} 71-4 leader primer TTTACCATGGAACATCTGTGGTTC (SEQ ID NO:15) contains a 5′ NcoI site (underlined). This leader primer (P-L) is used in conjunction with a second J_{H} primer for PCR amplification experiments. The 35 base pair J_{H} region oligonucleotide is designed to contain the same sequence for reverse priming at the 3′ end of the heavy chain variable gene, TTAGCGCGCTGAGGTGACCG

TGACC(A/G)(G/T)GGT (SEQ ID NO:16). This primer contains two degenerate positions with two nucleotides at each position. A BssH II site (left-underlined) 3′ to and immediately adjacent to the codon determining the last amino acid of the J region, allows convenient cloning at the 3′ end of the V_{H} gene. An internal BstE II site (right-underlined) is introduced as well. This sequence is used to amplify the V_{L} sequence. The fragments amplified by the P-L (leader primer) and P linker (reverse primer) and P-K (V₂ primer) and P-CK primers (reverse CK primer) are then cloned into an expression vector, such as the pRc/CMV (Invitrogen) and the resultant recombinant contains a signal peptide, V_{H} interchain linker and V_{L} sequences under the control of a promoter, such as the CMV promoter. The skilled artisan can readily choose other promoters that will express the gene in the cell system of choice, for example, a mammalian cell, preferably human cells.

To prepare anti-MHC-1 sFvs one could use the primer sequences A(SEQ ID NO:49) and B(SEQ ID NO:50) for V_{H}, C(SEQ ID NO:51) and D(SEQ ID NO:52) for V_{L}, which are set forth in Table 3. A preferred interchain linker for this antibody would be (gly-gly-gly-gly-ser)₃ (SEQ ID NO:1) and can readily be prepared by peptide synthesizers or excised and amplified by PCR from a plasmic containing this sequence. The sFv can be assembled from the various fragment (V_{H}, V_{L}, and interchain linker) by overlap extension [Horton, R. M., et al. Gene 77:61-68 (1989)] followed by amplification with primers SEQ ID NO:49 and SEQ ID NO:52. The complete sequence can be confirmed by the dideoxy chain termination method of Sanger [Proc. Natl. Acad. Sci. USA 74:5463-5467 (1977)].

Accordingly, as used herein the gene for the antibody can encompass genes for the heavy chain and light chain regions. In addition, the gene is operably linked to a promoter or promoters which results in its expression. Promoters that will permit expression in mammalian cells are well known and include cytomegalovirus (CMV) intermediate early promoter, a viral LTR such as the rous sarcoma virus LTR, HIV-LTR, HTLV-1 LTR, the simian virus 40 (SV40) early promoter, E. coli lac UV5 promoter and the herpes simplex tk virus promoter. This DNA sequence is described as the “antibody cassette”.

However, there are instances where a greater degree of intracellular specificity is desired. For example, as described above, when targeting MHC-1 molecules, it is desirable to direct the antibody to the ER. Thus, one preferably uses localization sequences in such instances. The antibodies can be delivered intracellularly and can be expressed there and bind to a target protein.

Localization sequences have been divided into routing signals, sorting signals, retention or salvage signals and membrane topology-stop transfer signals. [Pugsley, A. P., Protein Targeting, Academic Press, Inc. (1989)]. For example, in order to direct the antibody to a specific location, one can use specific localization sequences. For example, signals such as Lys Asp Glu Leu (SEQ ID NO:17) [Munro, et al., Cell 48:899-907 (1987)] Asp Asp Glu Leu (SEQ ID NO:18), Asp Glu Glu Leu (SEQ ID NO:19), Gln Glu Asp Leu (SEQ ID NO:20) and Arg Asp Glu Leu (SEQ ID NO:21) [Hangejorden, et al., J. Biol. Chem. 266:6015 (1991), for the endoplasmic reticulum; Pro Lys Lys Lys Arg Lys Val (SEQ ID NO:22) [Lanford, et al. Cell 46:575 (1986)] Pro Gln Lys Lys Ile Lys Ser (SEQ ID NO:23) [Stanton, L. W., et al., Proc. Natl. Acad. Sci USA 83:1772 (1986); Gln Pro Lys Lys Pro (SEQ ID NO:24) [Harlow, et al., Mol. Cell Biol. 5:1605 1985], Arg Lys Lys Arg (SEQ ID NO:55), for the nucleus; and Arg Lys Lys Arg Arg Gln Arg Arg Arg Ala His Gln (SEQ ID NO:25), [Seomi, et al., J. Virology 64:1803 (1990)], Arg Gln Ala Arg Arg Asn Arg Arg Arg Arg Trp Arg Glu Arg Gln Arg (SEQ ID NO:26) [Kubota, et al., Biochem. and Biophy, Res. Comm. 162:963 (1989)], Met Pro Leu Thr Arg Arg Arg Pro Ala Ala Ser Gln Ala Leu Ala Pro Pro Thr Pro (SEQ ID NO:27) [Siomi, et al., Cell 55:197 (1988)] for the nucleolar region; Met Asp Asp Gln Arg Asp Leu Ile Ser Asn Asn Glu Gln Leu Pro (SEQ ID NO:28), [Bakke, et al., Cell 63:707-716 (1990)] for the endosomal compartment. See, Letourneur, et al., Cell 69:1183 (1992) for targeting liposomes. Myristolation sequences, can be used to direct the antibody to the plasma membrane. In addition, as shown in Table 2 below, myristoylation sequences can be used to direct the antibodies to different subcellular locations such as the nuclear region. Localization sequences may also be used to direct antibodies to organelles, such as the mitochondria and the Golgi apparatus. The sequence Met Leu Phe Asn Leu Arg Xaa Xaa Leu Asn Asn Ala Ala Phe Arg His Gly His Asn Phe Met Val Arg Asn Phe Arg Cys Gly Gln Pro Leu Xaa (ID NO:29) can be used to direct the antibody to the mitochondrial matrix. (Pugsley, supra). See, Tang, et al., J. Bio. Chem. 207:10122, for localization of proteins to the Golgi apparatus.

TABLE 2

| AMINO- | | | |
|---|---|---|---|
| TERMINAL | SUBCELLULAR | | |
| SEQUENCE¹ | LOCATION² | PROTEIN | REFERENCE |
| GCVCSSNP | PM | p56^{USTRATCK} | Marchildon, et al. Proc. Natl. |
| (SEQ ID NO: 30) | | | Acad. Sci. USA 81: 7679-7682 |
| | | | (1984) |
| | | | Voronova, et al. Mol. Cell. |
| | | | Biol.4: 2705-2713 (1984) |
| GQTVTTPL | PM | Mul.V gag | Henderson, et al., Proc. Natl. |
| (SEQ ID NO: 31) | | | Acad. Sci. USA 80: 339-343 |
| | | | (1987) |
| GQELSQHE | PM | M-PMV gag | Rhee, et al, J. Virol. 61: 1045-1053 |
| (SEQ ID NO: 32) | | | (1987) |
| | | | Schultz, et al. J. Virol. 46: 355-361 |
| | | | (1983) |
| GNSPSYNP | PM | BLV gag | Schultz, et al., J. Virol. 133: 431-437 |
| (SEQ ID NO: 33) | | | (1984) |
| GVSGSKGQ | PM | MMTV gag | Schultz, et al. supra |
| (SEQ ID NO: 34) | | | |
| GQTITTPL | PM | FCL.V gag | Schultz, et al., supra |
| (SEQ ID NO: 35) | | | |
| GQTLTTPL | PM | BaEV gag | Schultz, et al. supra |
| (SEQ ID NO: 36) | | | |
| GQIFSRSA | PM | HTLV-I gag | Ootsuyama, et al., Jpn J. Cancer |
| (SEQ ID NO: 37) | | | Res. 76: 1132-1135 (1985) |
| GQIHGLSP | PM | HTLV-II gag | Ootsuyama, et al., supra |
| (SEQ ID NO: 38) | | | |
| GARASVLS | PM | HIV (HTLV- | Ratner, et al. Nature 313: 277-284 |
| (SEQ ID NO: 39 | | III) | (1985) |
| | | gag | |
| GCTLSAEE | PM | bovine brain Gₒ | Schultz, et al., Biochem. |
| (SEQ ID NO: 40) | | α-subunit | Biophys. Res. Commun. |
| | | | 146: 1234-1239 (1987) |
| GQNLSTSN | ER | Hepatitis B | Persing, et al., J. Virol. 61: 1672-1677 |
| (SEQ ID NO: 41) | | Virus pre-S1 | (1987) |
| GAALTILV | N | Polyoma Virus | Streuli, et al., Nature 326: 619-622 |
| (SEQ ID NO: 42) | | VP2 | (1987) |
| GAALTLLG | N | SV40 Virus | Streuli, et al., supra |
| (SEQ ID NO: 43) | | VP2 | |
| GAQVSSQK | S, ER | Poliovirus VP4 | Chow, et al., Nature 327: 482-486 |
| (SEQ ID NO: 44) | | | (1987) |
| | | | Paul, et al., Proc. Natl. Acad. |
| | | | Sci. USA 84: 7827-7831 (1987) |
| GAQLSRNT | S, ER | Bovine | Paul, et al., supra |
| (SEQ ID NO: 45) | | Enterovirus | |
| | | VP4 | |
| GNAAAAKK | G, S, N, C | cAMP- | Carr, et al., Proc. Natl. Acad. |
| (SEQ ID NO: 46) | | dependent | Sci. USA 79: 6128-6131 (1982) |
| | | kinase | |
| GNEASYPL | S, C | calcincurin B | Aitken, et al. |
| (SEQ ID NO: 47) | | | FEBS Lett. |
| | | | 150: 314-318 |
| | | | (1982) |
| GSSKSKPK | PM, C | P60^{SFC} | Schultz, et al., |
| (SEQ ID NO: 48) | | | Science 227: 427-429 (1985) |

¹To assist the reader, the standard single letter amino acid code is used in the Table, the amino acid sequences using the three letter code are set out in the Sequence Listing.
²Abbreviations are PM, plasma membranes, G. Golgi; N. Nuclear; C, Cytoskeleton; s, cytoplasm (soluble); M, membrane.

The antibody cassette is delivered to the cell by any of the known means. One preferred delivery system is described in U.S. patent application Ser. No. 08/199,070 by Marasco filed Feb. 22, 1994, which is incorporated herein by reference. This discloses the use of a fusion protein comprising a target moiety and a binding moiety. The target moiety brings the vector to the cell, while the binding moiety carries the antibody cassette. Other methods include, for example, Miller, A. D., Nature 357:455-460 (1992); Anderson, W. F., Science 256:808-813 (1992); Wu, et al, J. of Biol. Chem. 263:14621-14624 (1988). For example, a cassette containing these antibody genes, such as the sFv gene, can be targeted to a particular cell by a number of techniques. In the discussion below we will discuss the sFv genes coding for MHC-1 antibodies, which would be preferably introduced into human T-cells. Other delivery methods include the use of microcatheters, for example, delivering the vector in a solution which facilitates transfection, gene gun, naked DNA, adjuvant assisted DNA, liposomes, pox virus, herpes virus, adeno virus, retroviruses, etc.

In theory, there are multiple points within the secretory pathway at which an intrabody can be placed to bind and divert a trafficking protein from its ultimate destination. The ER is a preferred location because it permits trapping proteins early in their biosynthesis and creates potential for the rapid disposal of immune complexes by degradative systems within the ER [Klausner, R. D. & Sitia, R., Cell 62:611-614 (1990)]. Peptide signals required for the ER-retention of soluble proteins are well characterized and the carboxy terminal tetrapeptide Lys-Asp-Glu-Leu (KDEL) (SEQ ID NO.17) [Munroe, S. & Pehham, H. B., Cell 48:899-907 (1987)] is a preferred sequence. The efficiency of the ER retention system is in part due to the existence of a retrieval mechanism which returns KDEL-tagged (SEQ ID NO:1) proteins to the ER if and when they escape into the cis golgi network [Rothman, J. E. & Orci, L., Nature 355:409-415 (1992)]. The ER is also the natural site of antibody assembly as it is the residence to molecular chaperones such as BiP and GRP94, which assist in the correct folding of immunoglobulin molecules [Melnick, J., et al., Nature 370:373-375 (1994)]. The ER also offers the advantage that ER-resident proteins often show extended half-lives.

It will not in all instances be desired to knock out the receptor or peptide in all cells expressing it. Accordingly, in such instances, one preferably uses an inducible promoter, which is turned on predominantly in the cells you want to kill, for example, leukemic cells. For example, one can use a promoter that is induced by radiation to selectively turn on the desired cells. Another strategy to maximize the targeting of the specific cells is to use a delivery system, wherein the targeting moiety targets, for example, a second protein associated with the target cell.

The intrabodies bind to and form a complex with the molecules of interest intracellularly. By use of appropriate targeting signals, for example, the endoplasmic reticulum retention signal, such as KDEL (SEQ ID NO:17), one can further tailor the intrabodies. For example, one can prepare antibodies for MHC-1 (1) without any targeting signal (sFvMHC) and (2) with an endoplasmic reticulum retention signal (KDEL) (SEQ ID NO:17) (sFvMHCKDEL). Genes encoding these sFvs can then intracellularly inserted into mammalian cells.

Both intrabodies are expressed inside cells. However, the sFv MHC-1 KDEL intrabody is retained in the ER, whereas, the sFv MHC intrabody continues to move through the cell. As a consequence, the two intrabodies bind to and form complexes at different intracellular sites. For example, the ER intrabody (sFvMHCKDEL) binds and holds the receptor chain in the ER.

In some instances, where a total knockout of a receptor is desired, the use of IRES linked to a selectable marker, and strong promoter operably linked to the antibody is preferred. With certain receptors such as MHC-1, a total knock out can initiate an NK reaction. Thus, one preferably transfects such cells with a MHC-1 analog that is deficient in its ability to initiate an undesired immune reaction but will not initiate the NK reaction to avoid that reaction. For example, one such analog would be an MHC-1 molecule that lacks its cytoplasmic domain. Thus, the extracellular portion of the MHC-1 that the NK cells recognize would be present but the intracellular portion that signals and initiates the immune reaction would not be present. Other analogs that can accomplish this purpose can readily be prepared by one of ordinary skill in the art.

Using the above-described methodology, one can treat mammals, preferably humans, suffering from ailments caused by the expression of specific proteins, such as IRMs or antigens that produce an undesired immune response. For example, one can target the undesired antigens with an antibody that will specifically bind to such antigen. One delivers an effective amount of a gene capable of expressing the antibody, under conditions which will permit its intracellular expression, to cells susceptible to expression of the undesired target antigen. In other instances this method can be used as a prophylactic treatment to prevent or make it more difficult for such cells to be adversely affected by the undesired antigen, for example, by preventing processing of the protein and expression of the receptor. Where a number of targets exist, one preferred target is proteins that are processed by the endoplasmic reticulum. Intracellular delivery of any of the antibody genes can be accomplished by using procedures such as gene therapy techniques such as described above. The antibody can be any of the antibodies as discussed above. We discuss herein the use of this system to deliver antibody genes to T cells to alter an immune response, for example, the T cells of a mammal, for example, a human, in order to prepare for tissue transplantation or treat an autoimmune disease. However, it should be understood that based upon the present disclosure, one can readily adapt such an approach to other systems, for example, an individual with receptor abnormalities or to prevent an immune response to a particular antigen. In addition, this system can be used to transiently prevent receptor expression and thereby block undesired T-cell mediated reactions such as allograft rejections.

For certain cells, such as where the receptor, e.g., MHC-1 receptors, is vital for long-term survival, means are necessary to selectively administer the intrabody solely to aberrant cells. Numerous means exist as discussed above, including microcatheters, inducible promoters, and conjugates which enable selective administration of the intrabodies. For example, microcatheters can be used to deliver a solution containing the antibody cassette to the cells. Alternatively, the expression of the antibody can be controlled by an inducible promoter. Such a promoter could be activated by an effect of the target, or an outside source such as radiation. In such cells, malignant “cocktails” containing a mixture of antibodies can be used to target a number of receptors. In other cases, selection can lead to establishment of the cells that “turn-off” an intrabody, or no longer need the receptor for survival. With those cells the use of proteins at one time is desired because it makes it more difficult for mutants to evolve which will produce proteins capable of avoiding the antibody. Such “cocktails” can be administered together or by co-transfections. It is preferred that no more than about three proteins in the same intracellular region are targeted, preferably no more than about two. As long as another intracellular target is in a different cellular region, i.e., nucleus vs endoplasmic reticulum, it can also be targeted without having a detrimental effect on antibody production. This could be done using different localization sequences. If some target is not bound to the antibody at one location and, for instance, is further processed, it can be targeted at a subsequent location. Alternatively one could use multiple antibodies to target different epitopes of molecules.

Finally, antibody conjugates can be used to target aberrant cells. For example, genes can be delivered using a cell-specific gene transfer mechanism, which uses receptor-mediated endocytosis to carry RNA or DNA molecules into cells. For example, using an antibody against a receptor on the aberrant cell.

The antibodies that are used to target the cells can be coupled to a binding moiety to form an antibody-binding moiety by ligation through disulfide bonds after modification with a reagent such as succinimidyl-3-(2-pyridyldithio) proprionate (SPDP). The antibody-binding moiety complexes are produced by mixing the fusion protein with a moiety carrying the antibody cassette i.e. the DNA sequence containing the antibody operably coupled to a promoter such as a plasmid or vector. An alternative vector uses polyysine as a binding moiety.

As aforesaid, ligation with the antibodies can be accomplished using SPDP. First dithiopyridine groups will be introduced into both antibody or, for example, polylysine by means of SPDP and then the groups, e.g., in the polylysine can be reduced to give free sulfhydryl compounds, which upon mixing with the antibodies modified as described above, react to give the desired disulfide bond conjugates. These conjugates can be purified by conventional techniques such as using cation exchange chromatography. For example, a Pharmacia Mono S column, HR 10/10. These conjugates are then mixed with the antibody cassette under conditions that will permit binding. For example, incubating for one hour at 25° C. and then dialyzation for 24 hours against 0.15 M saline through a membrane with a molecular weight limit as desired. Such membranes can be obtained, for example, from Spectrum Medical Industries, Los Angeles, Calif.

Preferably the vectors of the present invention use internal ribosome entry site (IRES) sequences to force expression. As disclosed in Application No. 60/005,359, filed Oct. 16, 1995, the use of IRES allows the “forced-expression” of the desired gene, for example, an sFv. In another embodiment, one can use an IRES to force a stoichiometric expression of light chain and heavy chain, e.g., in a Fab. This forced expression avoids the problem of “silencing” where cells expressing the desired protein are phenotypically not seen, which may occur with a wide range of gene products. Another embodiment comprises using the IRES sequences the single chain intrabodies to the IRM of interest can be linked with a selectable marker. Selectable markers are well known in the art, e.g, genes that express protein that change the sensitivity of a cell to stimuli such as a nutrient, an antibiotic, etc. Examples of these genes include neo puro, tk, multiple drug resistance (MDR), etc.

The resultant products of that IRES linkage are not fusion proteins, and they exhibit their normal biological function. Accordingly, the use of these vectors permits the forced expression of a desired protein.

IRES sequences act on improving translation efficiency of RNAs in contrast to a promoter's effect on transcription of DNAs. A number of different IRES sequences are known including those from encephalomyocarditis virus (EMCV) [Ghattas, I. R., et al., Mol. Cell. Biol., 11:5848-5859 (1991); BiP protein [Macejak and Sarnow, Nature 353:91 (1991)]; the Antennapedia gene of drosophilia (exons d and e) [Oh, et al., Genes & Development, 6:1643-1653 (1992)]; as well as those in polio virus [Pelletier and Sonenberg, Nature 334: 320-325 (1988); see also Mountford and Smith, TIG 11, 179-184 (1985)].

IRES sequences are typically found in the 5′ noncoding region of genes. In addition to those in the literature they can be found empirically by looking for genetic sequences that effect expression and then determining whether that sequence effects the DNA (i.e. acts as a promoter or enhancer) or only the RNA (acts as an IRES sequence).

One can use these IRES sequences in a wide range of vectors ranging from artificial constructs (such as in U.S. Ser. No. 08/199,070, filed Feb. 22, 1994 to Marasco, et al.; PCT No. PCT/US95/02140) to DNA and RNA vectors. DNA vectors include herpes virus vectors, pox virus vectors, etc. RNA vectors are preferred. Still more preferably one uses a retroviral vector such as a moloney murine leukemia virus vector (MMLV) or a lentivirus vector such as HIV, SIV, etc. These vectors are sometimes referred to as defective vectors, and as used herein that term means that while the vectors retain the ability to infect, they have been altered so they will not result in establishment of a productive wild-type disease.

The forced expression vectors containing the sFvs to an IRM can be used in a variety of different systems ranging from in vitro to in vivo. For example, ex vivo studies can be performed on tissues, e.g., corneas or bone marrow, or cells which can be cultured. Thus, the present system is particularly useful with such cells, for example, with transforming bone marrow cells for transplantation. The present system can also be used in vivo as described above to prevent tissue transplant rejections, treat autoimmune diseases, etc.

The expression vectors can be used to transform cells by any of a wide range of techniques well known in the art, including electrophoresis, calcium phosphate precipitation, catheters, liposomes, etc.

To treat the targeted cells, these vectors can be introduced to the cells in vitro with the transduced cells injected into the mammalian host or the vector can be injected into a mammalian host such as a human where it will bind to, e.g., the T or B cell and then be taken up. To increase the efficiency of the gene expression in vivo, the antibody cassette can be part of an episomal mammalian expression vector. For example, a vector which contains the human Pappova virus (BK) origin of replication and the BK large T antigen for extra-chromosomal replication in mammalian cells, a vector which contains an Epstein-Barr (EB) virus origin of replication and nuclear antigen (EBNA-1) to allow high copy episomal replication. Other mammalian expression vectors such as herpes virus expression vectors, or pox virus expression vectors can also be used. Such vectors are available from a wide number of sources, including Invitrogen Corp. The antibody cassette is inserted into the expression vectors by standard techniques, for example, using a restriction endonuclease and inserting it into a specific site in such mammalian expression vector. These expression vectors can be mixed with the antibody-polylysine conjugates and the resulting antibody-polylysine-expression vector containing antibody cassette complexes can readily be made based upon the disclosure contained herein.

One would inject a sufficient amount of these vectors to obtain a serum concentration ranging between about 0.05 μg/ml to 20 μg/ml of antibody conjugate. More preferably between about 0.1 μg/ml to 10 μg/ml. Still more preferably, between about 0.5 μg/ml to 10 μg/ml.

These vectors can be administered by any of a variety of means, for example, parenteral injection (intramuscular (I.M.), intraperitoneal (I.P.), intravenous (I.V.), intracranial (I.C.) or subcutaneous (S.C.)), oral or other known routes of administration. Parenteral injection is typically preferred.

The materials can be administered in any convenient means. For example, it can be mixed with an inert carrier such as sucrose, lactose or starch. It can be in the form of tablets, capsules and pills. It can be in the form of liposomes or other encapsulated means. It can also be as part of an aerosol. For parenteral administration, it will typically be injected in a sterile aqueous or non-aqueous solution, suspension or emulsion in association with a pharmaceutically-acceptable parenteral carrier such as physiological saline.

Kits containing these materials in any of the above forms are also encompassed. Preferably, the kit contains instructions for the use of these intrabodies in accordance with the above teaching.

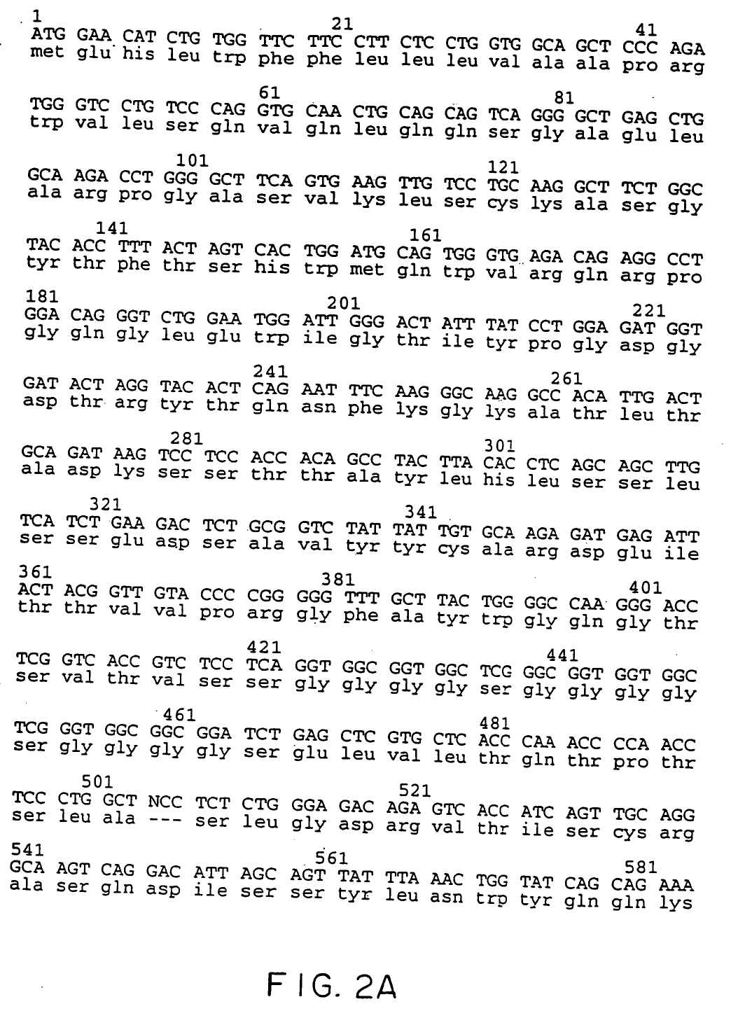

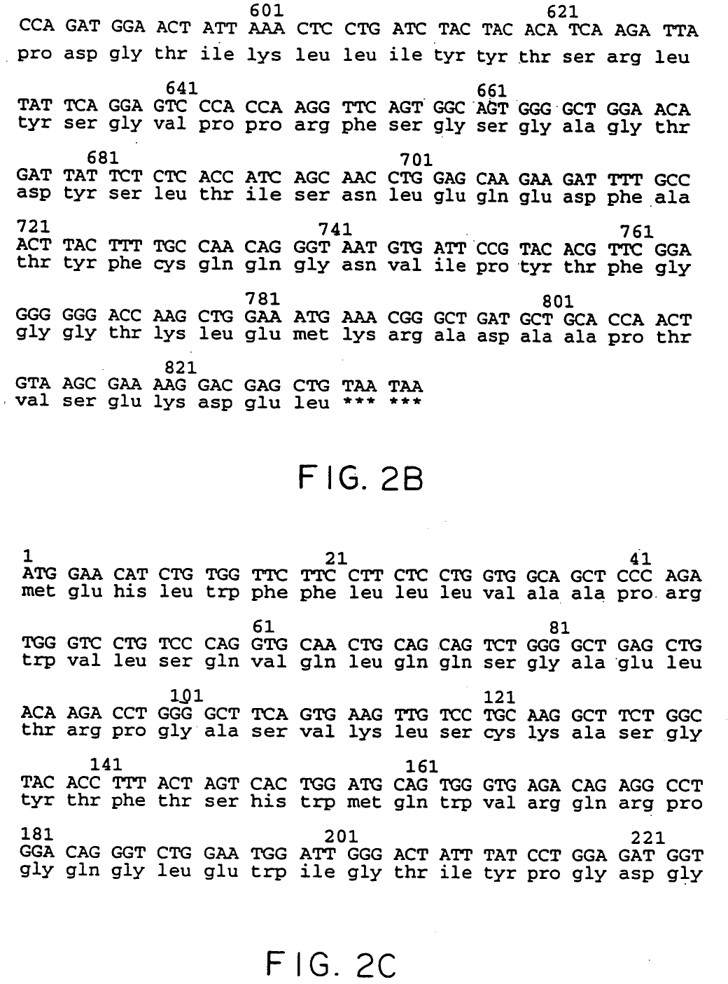

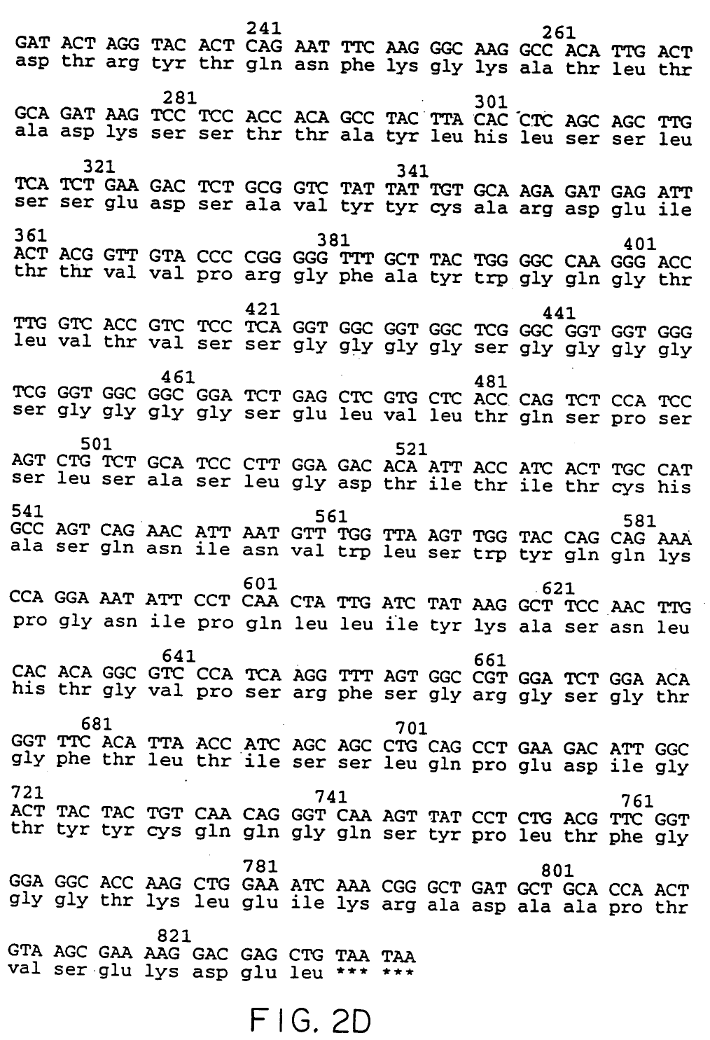

In one method of the present invention, we have produced ER-directed and KDEL containing sFv intrabodies to MHC-1 molecules. The 8k sFv is the molecule that is actually expressed in the hybridoma. In this case the heavy chain was promiscuous and anti-MHC-15k fragment could also be used (see FIGS. 2a and 2b). But the anti-MHC-1-8k is preferred and what is actually expressed in the cells.

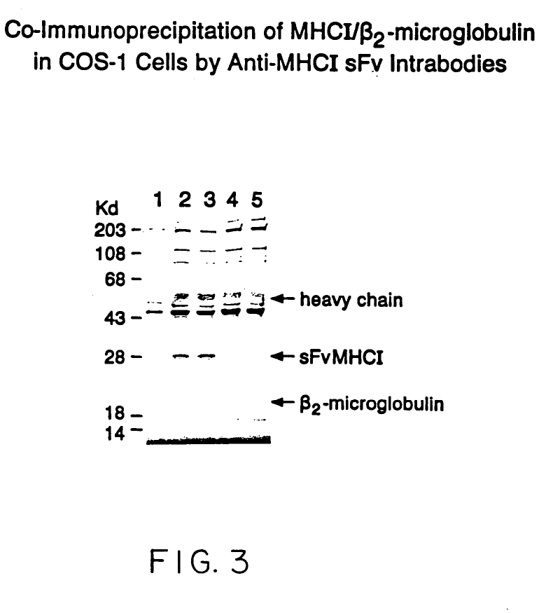

These constructs were cloned in prokaryotic and eukaryotic expression vectors (pHEN and pRc/CMV and pCMV4, respectively). Human CD4+T-lymphocyte cells were transfected with sFvhMHC-1 in pRc/CMV or pCMV4 vector. Cell surface expression of MHC-1 molecules was analyzed by immunofluorescent staining and Flow Cytometry. The results show that the sFv is being expressed (FIG. 3) and that the alpha and β2-microglobulin chains of the MHC-1 molecules is coimmunoprecipitable with the sFvMHC-1 molecules. Thus, the intrabody was expressed and able to bind its target intracellularly. We have also found that CD4+cells, constitutively expressing sFvhMHC-1 in the ER, effectively inhibited MHC-1 cell surface expression.

The present invention is further illustrated by the following examples, which are provided to aid in the understanding of the invention and are not construed as a limitation thereof.

### EXAMPLES

Construction of Endoplasmic Reticulum (ER) Expressed sFvhMHC-1

ER-directed and KDEL containing single-chain intrabodies against human MHC-1 were made using ATCC HB94 hybridoma cells (Fusion name BB7.7, anti-HLA-A, B, C) which reacts with combinatorial determinants of HLA-A,B,C and B-₂-microglobulin. The HB94 cells were used to isolate mRNA and cDNA.

Forward murine VH primer, 5′-cc-ctc-tag-aca-tat-gtg-aat-tcc-acc-atg-gcc-cag-gtc (SEQ ID NO: 49), and Reverse JH primer, 5′-tg(a/c)-gga-gac-ggt-gac-c(a/g)(a/t)-ggt-ccc-t (SEQ ID NO: 50), were used to amplify the Vk fragment. The VH and Vk fragments were linked via a (Gly₄Ser₁)₃ interchain-linker, using overlap-extension PCR [Clackson, T., et al, Nature 352:624-628 (1991)].

We isolated two specific Vkappa chains and so we had two series of sFvs, labeled as anti-MHC-1-5k and anti-MHC-1-8k sFvs, representing two different anti-MHC-sFvs with similar heavy chain and different kappa chains. Both had a C-terminal SEKDEL (SEQ ID NO:13) sequence specific for ER-retention. The nucleotide and amino-acid primary sequence is shown in FIGS. 2A and 2B (sFvhMHC-1-5k) and FIGS. 2C and 2D (sFvhMHC-1-8k).

The constructs were cloned in prokaryotic (pHEN) and eukaryotic (pRc/CMV and pCMV4) expression vectors according to the methods described in Mhashilkar, A. M., et al., Embo J 14:1542-1551 (1995).

The pHEN-constructs were used to isolate sFv protein from the periplasmic space of E. coli, and the pRc/CMV and pCMV4-constructs were used to analyze in-vitro transcription and translation, and produce transient and stably, sFvhMHC-1 expressing cells.

Cell Cultures

The human CD4⁺T-lymphocyte cell lines, SupT1 and Jurkat, were cultured in RPMI-1640 media supplemented with 10% fetal calf serum, glutamine (2 mM), penicillin-streptomycin (100 ug/mL) at 37° C. and 5% CO₂. The epithelial cell line, COS-1 cells, were grown in Dulbecco's modified Eagle's medium (DMEM) with 10% fetal calf serum and antibiotics.

Intracellular Expression of sFvhMHC-1 in Mammalian Cells.

The transient and constitutive expression of the various sFvhMHC-1 was detected an analyzed by immunoprecipitation and FACS analysis. The methods used were as follows:

Immunoprecipitation

10⁷ cells (either for transient transfection or stably expressing cells) were plated in 100 mm petriplates. For transient transfection the cells (COS-1) were plated at the above mentioned density 24 hours prior to transfection. DEAE-Dextran method of transfection was used [Fujita et al., Cell 46:401-407 (1986)]. In short, 10 μg of supercoiled plasmid DNA (sFvhMHC-1 in pRc/CMV or pCMV4 vector) was diluted with 1.8 mL of PBS and 100 μL of DEAE-Dextran (10 mg/mL stock made in water) was added to the mixture. The adherent cells were washed 2× with PBS prior to transfection.

DNA-DEAE-Dextran mix was layered on the cells and the plates were incubated at 37° C. for 30 min. The cells were reacted with chloroaquine (80 μM, final concentration) in 5 mL of serum-free DMEM media and let to incubate for another 2.5 hours at 37° C. The media was aspirated and replaced by 5 mL of fresh serum-free DMEM with 5% DMSO. After 2.5 minutes of further incubation, the media was drained and the cells were washed 2× with PBS and 7 mL of fresh 10% fetal-calf serum DMEM media was added and incubated until the cells were processed for metabolic labeling or exposed to neomycin selection for growing stable cells (48-60 hours post-transfection).

For immunoprecipitation, the transiently transfected or stable cell line was exposed to cysteine-free RPMI media (for 2 hours) and then metabolically labeled with 100-150 μCi of ³⁵S-cysteine. Cells were washed 3× with PBS and lysed with RIPA+ lysate buffer. Soluble proteins from the cell lysate were immunoprecipitated with rabbit-anti-mouse IgG (whole molecule, Sigma)-tagged Protein A sepharose beads. Proteins were resolved on 12.5% SDS-PAGE and visualized by autoradiography [Laemmli, U.K., Nature 227:680-685 (1970)].

Transfection (both transient and stable) of non-adherent T-lymphocytic cell lines (Sup T1 and Jurkat) was also done with DEAE-Dextran/Electroporation methods. In short, Cells were washed 3× with PBS and suspended in 0.8 mL of serumfree RPMI media to which 10 μg of plasmid DNA and 12.5 μL of DEAE-Dextran (10 mg/mL) was added. The DNA-DEAE-Dextran cells mixture was incubated for 30 minutes at 37° C. The cells were then washed 2× with serumfree RPMI and then plated with 10% fetal-calf serum in RPMI for 48-60 hours.

For electroporation we used BIORAD's Gene Pulser using the same amount of cells and pulsing them with 10 μgs of plasmid DNA (supercoiled/linearized) at settings of 250 volts, capacitance of 960 microfaradays for 18-24 seconds.

Transformed cells were then put in RPMI growth media, and 48-60 hours post-transfection, cells were either characterized for protein expression or exposed to neomycin selection. The concentration of neomycin in the liquid media for propagation of different stable cells lines were as follows: COS-1 cells, 500 μg/mL; Sup T1 cells, 400 μg/mL and Jurkat cells, 800 ug/mL.

FACS Analysis

Immunofluorescent staining was used to analyze cell surface expression of MHC-1 molecules in sFvhMHC-1 transduced/untransduced cells. Cells were washed 3× with PBS (with 1% Fetal Calf Serum), and incubated with HB94 hybridoma cells supernatant (1:50 dilution) for 2 hours at 4° C., following which the cells were washed 3× with PBS and then incubated with FITC-conjugated Rabbit anti-mouse IgG (1:500 dilution, Sigma) for 2 hours at 4° C. Cells were then washed 3× with PBS and resuspended in 0.4 mL of PBS with 4% formaldehyde.

The cells were then analyzed by Flow Cytometry in the Core-Facility of Dana Farber Cancer Institute.

Endoplasmic Reticulum (ER) expressed sFvhMHC-1.

Both the sFvhMHC-1-5k and 8k constructs had an open reading frame as observed by in-vitro transcription and translation method (Data not shown).

Transiently transfected COS-1 cells were analyzed for sFv expression using immunoprecipitation protocol as described earlier.

FIG. 3 illustrates the SDS-PAGE profile of sFvMHC-1s (lanes 2-5) and shows the transient expression of sFvMHC-1 in COS-1 cells. Radio-immunoprecipitation of transiently transfected, and metabolically radiolabeled cells were carried out using anti-mouse IgG (whole molecule, Sigma) bound Protein A-Sepharose. The samples were run on a 12.5% SDS-PAGE denaturing gel. Lane 1 is pRc/CMV vector control, Lanes 2&3 contain samples using two different plasmid preparations of pRc/CMV-sFvMHC-1-5k, Lanes 4 & 5 contain samples using two different plasmid preparation of pRc/CMV-sFvMHC-1-8k. In lanes 2-5, additional bands (′50 and 20 kD) are also co-immunoprecipitated.

A distinctive 30 kD band representing the sFv is observed. Also, two specific bands corresponding to 50 and 23 kD proteins are seen which could be the alpha and B₂ microglobulin chains of MHC-1 molecules being specifically pulled down with the sFvMHc-1 molecules (coimmunoprecipitable).

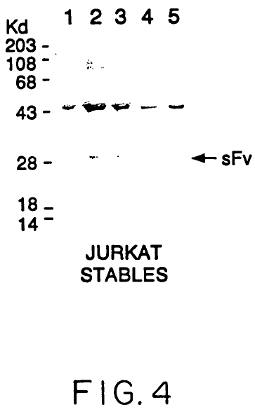

FIG. 4 shows stable cell expression of sFvhMHC-1 in Jurkat clones under Neomycin selection. Neomycin selected, stable sub-clones of sFvMHC-1 expressing Jurkat cells, were analyzed for intrabody expression. Lane 1 contains pRc/CMV vector clone. Lanes 2 & 3 contain sFvhMHC-1-5k stable subclones. Lanes 4 & 5 contain sFvhMHC-1-8k stable subclones. The result show a sFv band of 30 kD.

Downregulation of Cell Surface MHC-1 Expression in sFvhMHC-1 Stable Cells.

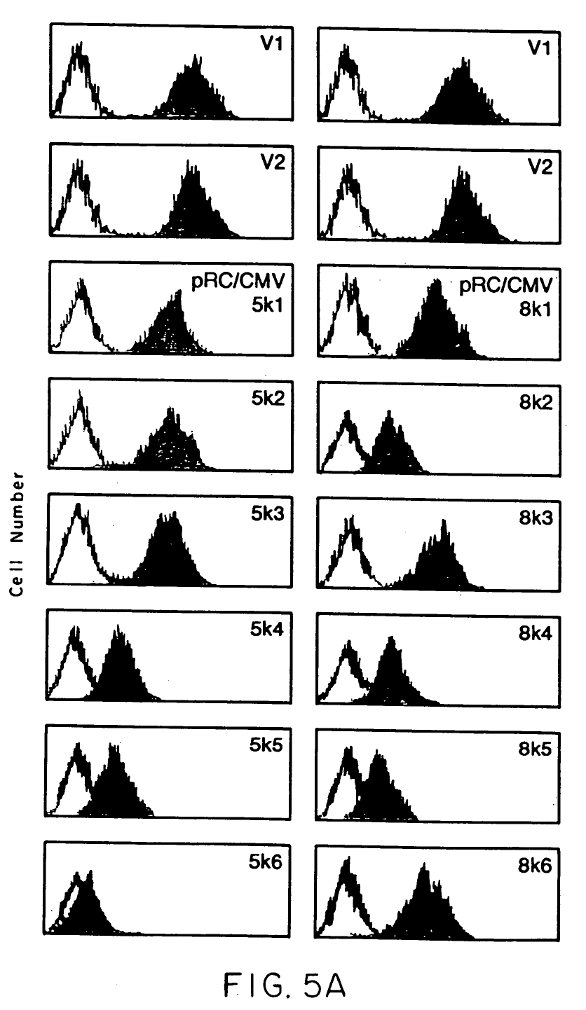

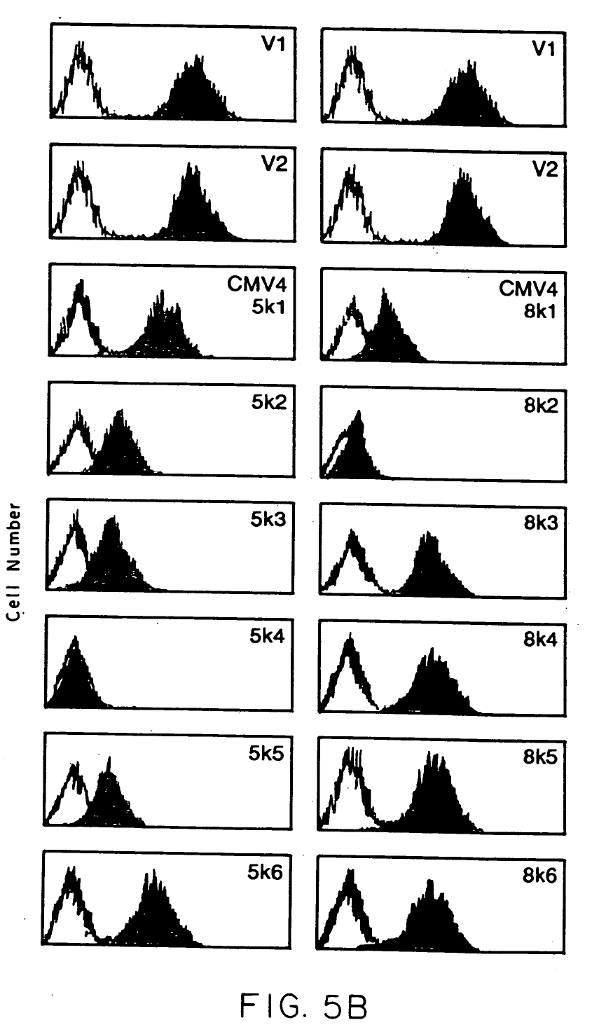

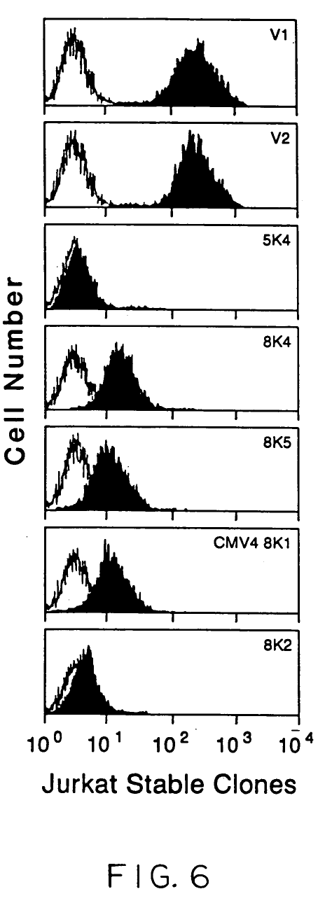

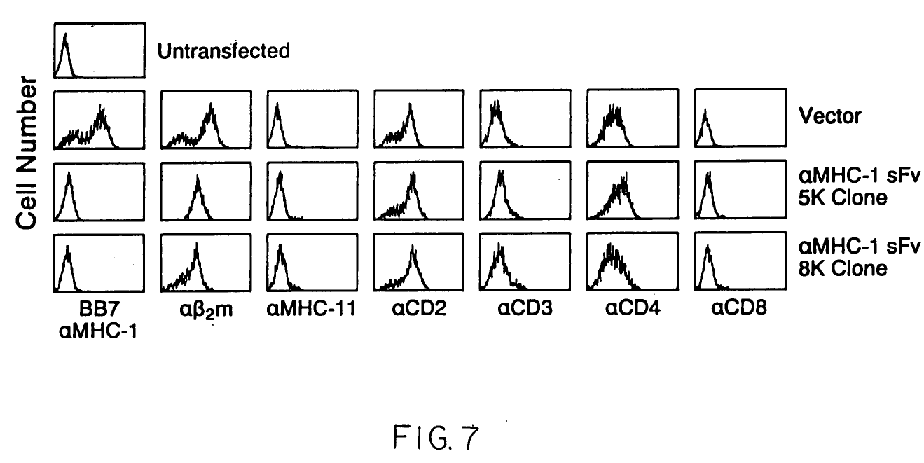

FIGS. 5 and 6 show stable, sFvhMHC-1 expressing, Jurkat subclones that show different levels of MHC-1 downmodulation using either sFv5k or sFv8k under pRc/CMV or pCMV4 control.

FIG. 5 shows FACS analysis of Jurkat stable subclones. Jurkat cells expressing sFvMHC-1 or empty vectors were incubated first with HB94 hybridoma supernatant, followed by a FITC-labeled anti-mouse IgG (Sigma). These cells were monitored for MHC-1 cell surface expression. Column 1 shows pRc/CMV-vector alone or sFvhMHC-1-5k subclones. Column 2 shows pRc/CMV-vector alone or sFvhMHC-1-8k subelones. Column 3 shows pCMV4-vector alone or sFvhMHC-1-5k subclones. Column 4 shows pCMV4-vector alone or sFvMHC-1-8k subclones. These results show that MHC-1 receptor expression is inhibited by the sFvMHC-1-8k intrabody. FIG. 5 shows the variability in phenotypic knock-out observed in different subclones. For example, there is almost complete knockout in subclones pRC/CMV/5k6, CMV4/5k4 and CMV4/8k2.

FIG. 6 shows the FACS analysis of selected Jurkat stable subclones. FIG. 6 shows that clone 5k under pRc/CMV control and clone 8k under CMV4 control are devoid of or show a minimal amount of MHC-i expression, respectively.

FIG. 7 shows FACS analysis of one pRc/CMV empty vector and two sFvhMHC-1 subclones. Cell surface expression levels of MHC-1, MHC-2, B2-microglobulin, CD2, CD3, CD4 and CD8 were analyzed or vector alone transformed subclone and two sFvhMHC-1 transformed clones. FIG. 7 shows a panel of the two clones in parallel with a vector control, demonstrating the other different surface markers present on them, which included MHC-1 (whole molecule), B2-microglobulin, MHC-2, CD2, CD3, CD4 and CD8. These results show the downregulation of the MHC-1 molecules compared to the vector control, while the other surface receptors remain unaffected, as compared with the vector control. It appears that β2-microglobulin gets through to the surface, perhaps due to a non-classical pathway which is independent of the MHC-1 molecule.

These studies demonstrate that CD4+Jurkat cells, constitutively expressing sFvhMHC-1 in ER, effectively inhibited MHC-1 cell surface expression, and using these sFvs we were able to coimmunoprecipitate MHC-1 alpha chain and B2-microglobulin.

Retroviral Infection

We have cloned sFvhMHC-1 in the Murine Maloney retroviral LN vector [Miller, A. D., Immunology vol. 158 (1994)].

The retroviral construct is transduced in the ecotropic cell line oCRE. After 48 h, supernatants is used to infect packaging cell line PA317. Producer cell lines is established following G418 and HAT selection. Initial screening is performed to ensure sFv expression from the recombinant viruses. The supernatants of G418 resistant cells is used to infect immortalized T-lymphocytes and stimulated PBLs. Protein expression in the transduced cell lines is examined by immunofluorescence, immunoprecipitation and ELISA. A cell line transduced with vector control (without sFv) and an irrelevant sFv (sFvtac) is used in parallel and analyzed.

All the references mentioned herein are incorporated by reference.

The invention has been described in detail with particular reference to the preferred embodiments thereof. However, it will be appreciated that modifications and improvements within the spirit and teachings of this inventions may be made by those in the art upon considering the present disclosure.

We claim:

1. A method of inhibiting an undesired MHC class 1 immune associated reaction comprising transducing a cell that can be involved in the undesired MHC class 1 immune associated reaction with a first gene encoding an antibody, wherein said antibody when expressed will bind in the cell to a target molecule involved in the undesired MHC class 1 immune associated reaction, expressing the antibody and letting said antibody bind to said target molecule, and also transducing said cell with a second gene encoding an MHC-1 analog that is deficient in its ability to initiate an MHC class 1 reaction, wherein said MHC-1 analog will not initiate the NK (Natural Killer) reaction.

2. The method of claim 1, wherein the antibody comprises a single chain antibody.

3. The method of claim 1, wherein said antibody binds to an MHC Class I component selected from the group consisting of MHC Class Iα chains, β2 microglobulin, calnexin, transporter associated with antigen processing (TAP) and tapasin.

4. The method of claim 1, wherein the target molecule is an MHC class I molecule, and the undesired immune reaction is tissue rejection during transplantation.

5. The method of claim 4, wherein the cell is an antigen presenting cell.

6. The method of claim 1, wherein the gene encoding the antibody is in an RNA or DNA vector.

7. The method of claim 6, wherein the gene encoding the antibody is in a DNA vector.

8. The method of claim 1, wherein the MHC-1 analog is an MHC-1 molecule that lacks its cytoplasmic domain.
